(19) United States
(12) Patent Application Publication
(10) Pub. No.: US 2025/0179173 A1
(43) Pub. Date: Jun. 5, 2025

(54) COMBINATION THERAPY OF LILRB ANTAGONIST AND PD-1/PD-L1 AXIS INHIBITOR

(71) Applicant: IMMUNE-ONC THERAPEUTICS, Inc., Palo Alto, CA (US)

(72) Inventors: X. Charlene LIAO, Palo Alto, CA (US); J. Paul WOODARD, Palo Alto, CA (US); Luke CHUNG, Palo Alto, CA (US); Wen Hong LIN, Palo Alto, CA (US); Tao HUANG, Palo Alto, CA (US); Maria Jose COSTA, Palo Alto, CA (US); Juanhong GU, Palo Alto, CA (US); Hong XIANG, Palo Alto, CA (US)

(21) Appl. No.: 18/839,014

(22) PCT Filed: Feb. 17, 2023
(86) PCT No.: PCT/US2023/062778

§ 371 Date: Aug. 15, 2024

Related U.S. Application Data
(60) Provisional application No. 63/311,446, filed on Feb. 17, 2022.

(51) Int. Cl. C07K 16/28; A61P 35/00

(52) U.S. Cl. CPC C07K 16/2803; A61P 35/00; C07K 16/2827; C07K 2317/24

(57) ABSTRACT

The present disclosure provides herein monotherapies of a LILRB antagonist (e.g., an anti-LILRB2 antibody or anti-LILRB4 antibody) and combination therapies of the LILRB antagonist (e.g., an anti-LILRB2 antibody or anti-LILRB4 antibody) and a PD-1/PD-L1 axis inhibitor for cancer patients. Wherein the antagonist of LILRB is an anti-LILRB1 antibody, an anti-LILRB2 antibody, an anti-LILRB3 antibody, an anti-LILRB4 antibody, an anti-LILRB5 antibody or an anti-LAIR1 antibody.

### CROSS-REFERENCE

This application claims priority to U.S. provisional patent applications No. 63/311,446, filed Feb. 17, 2022, the disclosure of which is incorporated herein by reference.

### SEQUENCE LISTING

The sequence listing that is contained in the file named “066564-8017WO01-sequence list-FINAL-updated.xml”, which is 21,735 bytes (as measured in Microsoft Windows) and was created on Feb. 17, 2023, is filed herewith by electronic submission and is incorporated by reference herein.

### FIELD OF THE INVENTION

The present disclosure generally relates to combination therapy involving a LILRB antagonist and a PD-1/PD-L1 axis inhibitor to treat solid tumors and hematologic malignancies.

### BACKGROUND

The LILRB family of receptors are type I transmembrane glycoproteins having 1) extracellular Ig-like domains (which bind to ligands) and 2) intracellular immunoreceptor tyrosine-based inhibitory motifs (ITIMs) (which can recruit phosphatases SHP-1, SHP-2, or SHIP to negatively regulate immune cell activation); hence, the members of the LILRB family of receptors are classified as immune inhibitory receptors. LILRBs have been known to be expressed on myeloid cells and certain other hematopoietic cells. LILRBs have shown to inhibit activities of a number of immune cell types, thereby promoting tolerance and facilitating tumor immune escape.

Several antibodies against LILRBs have been developed for treating LILRB-expressing cancers and solid tumors infiltrated by LILRB-expressing leukocytes. However, significant need exists for combination therapies for LILRB-expressing cancers and for solid tumors infiltrated by LILRB-expressing leukocytes to meet clinical needs.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides, among others, a method of treating cancer in a subject in need thereof, comprising:
administering to the subject a therapeutically effective amount of an antagonist of LILRB (e.g., an anti-LILRB2 antibody, an anti-LILRB4 antibody), optionally in combination with a therapeutically effective amount of an inhibitor of PD-1/PD-L1 axis, such that the subject has stable disease, partial response, or complete response for at least 3 months (e.g., 4 months, 5 months, 6 months, 7 months, 8 months, 9 months, or 10 months).

In certain embodiments, the method comprises administering the antagonist of LILRB and optionally the PD-1/PD-L1 axis inhibitor with a dosing interval of once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In certain embodiments, the LILRB is LILRB1, LILRB2, LILRB3, LILRB4, LILRB5 or LAIR1.

In certain embodiments, the antagonist of LILRB is an anti-LILRB specific antibody, such as a monoclonal anti-LILRB antibody, a bi-specific antibody targeting LILRB and a second antigen, a bi-specific antibody targeting two LILRBs, an antibody-drug conjugate targeting one or more LILRBs or an immune cell expressing a chimeric antigen receptor (CAR) or a genetically modified TCR comprising an anti-LILRB antigen binding domain. In certain embodiments, the antibody disclosed herein has ADCC activities or enhanced ADCC activities.

In certain embodiments, the antagonist of LILRB is an anti-LILRB1 antibody, an anti-LILRB2 antibody, an anti-LILRB3 antibody, an anti-LILRB4 antibody, an anti-LILRB5 antibody or an anti-LAIR1 antibody.

In certain embodiments, the anti-LILRB1 antibody is an antibody disclosed in PCT/US2021/043128 (published as WO2022026360).

In certain embodiments, the anti-LILRB2 antibody is an antibody disclosed in PCT/US2021/015362 (published as WO2021158413) or PCT/US2021/055927.

In certain embodiments, the anti-LILRB2 antibody:
a) comprises a constant region of human IgG4 having a mutation of S228P;
b) is capable of specifically binding to LILRB2 at an EC50 value of less than 2 nM (e.g., about 1.5 nM, about 1.4 nM, about 1.3 nM, about 1.2 nM, about 1.1 nM or about 1.0 nM) as measured by flow cytometry, ELISA, or reporter gene expression;
c) is capable of blocking binding of LILRB2 to one or more ligands selected from the group consisting of HLA-G, classical MHC-I, ANGPTLs (e.g., ANGPTL2), CSPs, SEMA4A and CD1d;
d) is capable of specifically binding to LILRB2 with a binding affinity as measured by bio-layer interferometry of less than 20 nM (e.g., about 18 nM, 15 nM, 10 nM, 5 nM, 2 nM, 1.5 nM, about 1.4 nM, about 1.3 nM, about 1.2 nM, about 1.1 nM or about 1.0 nM); or
e) has an off-rate after binding to LILRB2 at a koff value of less than 5×10⁻⁴ (e.g., about 4×10⁻⁴, about 3×10⁻⁴)/s as measured by bio-layer interferometry.

In certain embodiments, the anti-LILRB2 antibody comprises the same heavy chain complementary determining regions (HCDRs) and the same light chain CDRs (LCDRs) as the HCDRs and the LCDRs of the anti-LILRB2 antibody B2-19-16, wherein the anti-LILRB2 antibody B2-19-16 comprises a heavy chain variable region comprising the amino acid sequence of SEQ ID NO: 7, and a light chain variable region comprising the amino acid sequence of SEQ ID NO: 8.

In certain embodiments, the anti-LILRB2 antibody comprises a heavy chain variable region comprising HCDR1, HCDR2 and HCDR3, and a light chain variable region comprising LCDR1, LCDR2 and LCDR3, wherein the HCDR1 comprises an amino acid sequence of SEQ ID NO: 1, the HCDR2 comprises an amino acid sequence of SEQ ID NO: 2, the HCDR3 comprises an amino acid sequence of SEQ ID NO: 3, the LCDR1 comprises an amino acid sequence of SEQ ID NO: 4, the LCDR2 comprises an amino acid sequence of SEQ ID NO: 5, and the LCDR3 comprises an amino acid sequence of SEQ ID NO: 6.

In certain embodiments, the anti-LILRB2 antibody comprises a heavy chain variable region comprising the amino acid sequence of SEQ ID NO: 7, and a light chain variable region comprising the amino acid sequence of SEQ ID NO: 8.

In certain embodiments, the anti-LILRB2 antibody further comprises a constant region of human IgG4 having a mutation of S228P.

In certain embodiments, the anti-LILRB2 antibody is administered Q3W at a dosage of about 60 mg to about 1800 mg, about 800 mg to about 1600 mg, about 1000 mg to about 1400 mg, or about 1200 mg, or with weekly equivalent amount for any dosing interval from once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In certain embodiments, the anti-LILRB2 antibody is administered at a dosage of about 60 mg to about 1800 mg Q3W.

In certain embodiments, the anti-LILRB2 antibody is administered at a dosage of about 1200 mg Q3W.

In certain embodiments, the anti-LILRB2 antibody is administered intravenously (IV).

In certain embodiments, the anti-LILRB2 antibody is administered subcutaneously (SC).

In certain embodiments, the anti-LILRB3 antibody is an antibody disclosed in PCT/US2021/061630.

In certain embodiments, the anti-LILRB4 antibody is an antibody disclosed in U.S. Pat. No. 10,501,538, US20210371518, or PCT/US2021/022029 (published as WO2021183839).

In certain embodiments, the anti-LILRB4 antibody:
a) comprises a constant region of human IgG1 with a functional Fc;
b) comprises a constant region of human IgG4;
c) is capable of specifically binding to human LILRB4 with a binding affinity of less than 1 nM (e.g., less than 0.9 nM, less than 0.8 nM, less than 0.7 nM, less than 0.6 nM, less than 0.5 nM, less than 0.4 nM, less than 0.3 nM, less than 0.2 nM or less than 0.1 nM) as measured by bio-layer interferometry;
d) is capable of blocking APOE binding to LILRB4 at an IC50 value of less than 0.5 nM (e.g., less than 0.4 nM, less than 0.3 nM, less than 0.2 nM or less than 0.1 nM) as measured by flow cytometry, ELISA, or reporter gene expression; or
e) is capable of blocking Fibronectin binding to LILRB4 at an IC50 value of less than 1 nM (e.g., less than 0.9 nM, less than 0.8 nM, less than 0.7 nM, less than 0.6 nM, less than 0.5 nM, less than 0.4 nM, less than 0.3 nM, less than 0.2 nM or less than 0.1 nM) as measured by flow cytometry, ELISA, or reporter gene expression.

In certain embodiments, the anti-LILRB4 antibody comprises the same heavy chain complementary determining regions (HCDRs) and the same light chain CDRs (LCDRs) as the HCDRs and the LCDRs of the anti-LILRB4 antibody H7K3m5, wherein the anti-LILRB4 antibody H7K3m5 comprises a heavy chain variable region comprising the amino acid sequence of SEQ ID NO: 15, and a light chain variable region comprising the amino acid sequence of SEQ ID NO: 16.

In certain embodiments, the anti-LILRB4 antibody comprises a heavy chain variable region comprising HCDR1, HCDR2 and HCDR3, and a light chain variable region comprising LCDR1, LCDR2 and LCDR3, wherein the HCDR1 comprises an amino acid sequence of SEQ ID NO: 9, the HCDR2 comprises an amino acid sequence of SEQ ID NO: 10, the HCDR3 comprises an amino acid sequence of SEQ ID NO: 11, the LCDR1 comprises an amino acid sequence of SEQ ID NO: 12, the LCDR2 comprises an amino acid sequence of SEQ ID NO: 13, and the LCDR3 comprises an amino acid sequence of SEQ ID NO: 14.

In certain embodiments, the anti-LILRB4 antibody comprises a heavy chain variable region comprising the amino acid sequence of SEQ ID NO: 15, and a light chain variable region comprising the amino acid sequence of SEQ ID NO: 16.

In certain embodiments, the anti-LILRB4 antibody is administered Q3W at a dosage of about 250 mg to about 2400 mg, about 300 mg to about 2200 mg, about 350 mg to about 2000 mg, about 400 mg to about 1800 mg, about 450 mg to about 1600 mg, about 500 mg to about 1400 mg, about 550 mg to about 1200 mg, about 600 mg to about 1000 mg, about 650 mg to about 800 mg, or about 700 mg, or with weekly equivalent amount for any dosing interval from once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In certain embodiments, the anti-LILRB4 antibody is administered at a dosage of about 60 mg to about 2400 mg Q3W.

In certain embodiments, the anti-LILRB4 antibody is administered at a dosage of about 800 mg Q3W.

In certain embodiments, the anti-LILRB4 antibody is administered intravenously (IV).

In certain embodiments, the anti-LILRB4 antibody is administered subcutaneously (SC).

In certain embodiments, the anti-LAIR1 antibody is an antibody disclosed in US20190338026.

In certain embodiments, the anti-LILRB antibody is an antibody disclosed in U.S. Ser. No. 16/321,745 (published as US20210349096A).

In certain embodiments, the subject is determined to have PD-L1 expression in the diseased tissue, e.g., tumor tissues, or the diseased cell, e.g., malignant or cancer cells. In certain embodiments, the subject is determined to have PD-L1 expression in immune cells, e.g., myeloid cells, NK cells, B cells, plasma cells, plasmablasts, or T-cells that infiltrate the tumor tissues.

In certain embodiments, the subject is determined to have stable disease (SD) for more than 3, 4, 5, 6, 7, or 8 months (preferably 6 months).

In certain embodiments, the subject has received one or more (e.g., 1, 2, 3, 4, 5, 6, 7, 8, 9, 10, 11, 12, 13, 14, or 15) prior lines of therapies.

In certain embodiments, the subject has received one or more (e.g., 1, 2, 3, 4, 5, 6, 7, 8, 9, 10, 11, 12, 13, 14, or 15) prior lines of immunotherapy or immuno-oncology therapies.

In certain embodiments, the subject has not received any prior line of immunotherapy or immuno-oncology therapy.

In certain embodiments, the subject has received chemotherapy.

In certain embodiments, the inhibitor of PD-1/PD-L1 axis is a PD-1 inhibitor.

In certain embodiments, the PD-1 inhibitor is selected from Tables 1-2.

In certain embodiments, the PD-1 inhibitor is cemiplimab or tislelizumab.

In certain embodiments, the inhibitor of PD-1/PD-L1 axis is an PD-L1 inhibitor.

In certain embodiments, the PD-L1 inhibitor is selected from Tables 3-4.

In certain embodiments, the PD-L1 inhibitor is atezolizumab, durvalumab or avelumab.

In certain embodiments, the subject is human.

In certain embodiments, the administration is via oral, nasal, intravenous, subcutaneous, sublingual, intra-tumoral or intramuscular administration.

In certain embodiments, the administration of the composition comprising anti-LILRB antibody (e.g., an anti-LILRB2 antibody, an anti-LILRB4 antibody) is prior to, simultaneously with, or after the administration of the composition comprising PD-1/PD-L1 axis inhibitor.

In certain embodiments, the disease or condition is cancer.

In certain embodiments, the cancer cell is a cell from bladder, blood, bone, bone marrow, brain, breast, colon, endometrium, esophagus gallbladder, gastrointestine, gum, head, kidney, liver, lung, nasopharynx, neck, ovary, prostate, skin, soft tissue, stomach, pancreas, testis, thyroid, tongue, cervix, or uterus.

In certain embodiments, the cancer is selected from the group consisting of gastric cancer, lung cancer (e.g., non-small-cell lung carcinoma (NSCLC)), bronchial cancer, bone cancer, bile duct cancer, cholangiocarcinoma, pancreatic cancer, breast cancer (e.g., ER+HER2− breast cancer, triple-negative breast cancer (TNBC)), liver cancer (e.g., HCC), ovarian cancer, testicle cancer, kidney cancer (e.g., renal cell carcinoma (RCC), such as clear cell renal cell carcinoma (ccRCC)), bladder cancer, head and neck cancer (e.g., Head and neck squamous cell carcinoma (HNSCC)), nasopharyngeal cancer, spine cancer, brain cancer, cervix cancer, uterine cancer, endometrial cancer, colon cancer (e.g., MSI-H cancer), colon neuroendocrine, rectal cancer, anal cancer, esophageal cancer (e.g., esophageal squamous cell carcinoma (ESCC)), gastrointestinal cancer, Merkel cell carcinoma, skin cancer, prostate cancer, pituitary cancer, stomach cancer, vagina cancer, thyroid cancer, adrenal carcinoid tumor, glioblastoma, astrocytoma, melanoma, disseminated cancer unknown primary cancer, myelodysplastic syndrome, sarcoma, teratoma, adenocarcinoma, high microsatellite instability (MSI-H)/mismatch repair deficient (dMMR) tumors (e.g., MSI-H/dMMR CRC) and small intestine cancer.

In certain embodiments, the cancer is selected from the group consisting of: merkel cell carcinoma, cholangiocarcinoma, colon neuroendocrine, rectal cancer, colon cancer, pancreatic cancer, HNSCC, breast cancer (e.g., ER+Her2− breast cancer), adrenal carcinoid tumor, bladder cancer, ovarian cancer, disseminated cancer unknown primary cancer, MSI-H/dMMR CRC, RCC, gastric cancer, NSCLC, TNBC and small intestine cancer.

In another aspect, also provided herein is a kit useful in treating a disease or condition in a subject in need thereof, comprising a first container that comprises a LILRB antagonist (e.g., an anti-LILRB2 antibody, an anti-LILRB4 antibody) and a second container that comprises a PD-1/PD-L1 axis inhibitor, and optionally instructions for use of the kit.

In another aspect, also provided herein is a kit useful in treating a disease or condition in a subject in need thereof, comprising a first container that comprises a LILRB antagonist (e.g., an anti-LILRB2 antibody, an anti-LILRB4 antibody) and a second container that comprises a PD-1/PD-L1 axis inhibitor, and optionally instructions for co-administration of a LILRB antagonist and a PD-1/PD-L1 axis inhibitor.

In another aspect, also provided herein is a kit, comprising a LILRB antagonist (e.g., an anti-LILRB2 antibody, an anti-LILRB4 antibody) and a package insert comprising instructions for using the LILRB antagonist (e.g., an anti-LILRB2 antibody, an anti-LILRB4 antibody) in combination with a PD-1/PD-L1 axis inhibitor to treat a disease or condition in a subject in need thereof.

In another aspect, also provided herein is a kit, comprising a PD-1/PD-L1 axis inhibitor and a package insert comprising instructions for using the PD-1/PD-L1 axis inhibitor in combination with a LILRB antagonist (e.g., an anti-LILRB2 antibody, an anti-LILRB4 antibody) to treat a disease or condition in a subject in need thereof.

In certain embodiments, the instruction comprises a dosing regimen for administering the LILRB antagonist with a dosing interval of once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In certain embodiments, the instruction comprises a dosing regimen for administering the LILRB antagonist Q3W with a dosage of about 50 mg, about 100 mg, about 150 mg, about 200 mg, about 250 mg, 300 mg, about 350 mg, about 400 mg, about 450 mg, about 500 mg, about 550 mg, about 600 mg, about 700 mg, about 800 mg, about 1000 mg, about 1200 mg, about 1400 mg, about 1600 mg, or about 1800 mg, or with weekly equivalent amount for any dosing interval from once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In certain embodiments, the instruction comprises a dosing regimen for administering the anti-LILRB2 antibody at a dosage of about 60 mg to about 1800 mg Q3W, about 800 mg to about 1600 mg Q3W, about 1000 mg to about 1400 mg Q3W, or about 1200 mg Q3W.

In certain embodiments, the instruction comprises a dosing regimen for administering the anti-LILRB4 antibody Q3W at a dosage of about 250 mg to about 2400 mg, about 300 mg to about 2200 mg, about 350 mg to about 2000 mg, about 400 mg to about 1800 mg, about 450 mg to about 1600 mg, about 500 mg to about 1400 mg, about 550 mg to about 1200 mg, about 600 mg to about 1000 mg, about 650 mg to about 800 mg, or about 700 mg, or with weekly equivalent amount for any dosing interval from once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In certain embodiments, the instruction comprises a dosing regimen for administering the anti-LILRB4 antibody at a dosage of about 60 mg to about 2400 mg Q3W, about 250 mg to about 2400 mg Q3W, about 800 mg to about 2400 mg Q3W, or about 800 mg to 1200 mg Q3W.

In certain embodiments, the anti-LILRB2 or anti-LILRB4 antibody is administered intravenously (IV).

In certain embodiments, the anti-LILRB2 or anti-LILRB4 antibody is administered subcutaneously (SC).

In certain embodiments, the disease or condition is a cancer selected from the group consisting: gastric cancer, lung cancer (e.g., non-small-cell lung carcinoma (NSCLC)), bronchial cancer, bone cancer, liver and bile duct cancer, cholangiocarcinoma, pancreatic cancer, breast cancer (e.g., ER+HER2− breast cancer, triple-negative breast cancer (TNBC)), liver cancer (e.g., HCC), ovarian cancer, testicle cancer, kidney cancer (e.g., renal cell carcinoma (RCC), such as clear cell renal cell carcinoma (ccRCC)), bladder cancer, head and neck cancer (e.g., Head and neck squamous cell carcinoma (HNSCC)), nasopharyngeal cancer, spine cancer, brain cancer, cervix cancer, uterine cancer, endometrial cancer, colon cancer (e.g., MSI-H cancer), colon neuroendocrine, rectal cancer, anal cancer, esophageal cancer (e.g., esophageal squamous cell carcinoma (ESCC)), gastrointestinal cancer, Merkel cell carcinoma, skin cancer, prostate cancer, pituitary cancer, stomach cancer, vagina cancer, thyroid cancer, adrenal carcinoid tumor, glioblastoma, astrocytoma, melanoma, disseminated cancer unknown primary cancer, myelodysplastic syndrome, sarcoma, teratoma, adenocarcinoma and high microsatellite instability (MSI-H)/mismatch repair deficient (dMMR) tumors (e.g., MSI-H/dMMR CRC).

In certain embodiments, the cancer is selected from the group consisting: Merkel cell carcinoma, cholangiocarcinoma, colon neuroendocrine, rectal cancer, colon cancer, pancreatic cancer, HNSCC, breast cancer (e.g., ER+Her2− breast cancer), adrenal carcinoid tumor, bladder cancer, ovarian cancer, disseminated cancer unknown primary cancer, MSI-H/dMMR CRC, RCC, gastric cancer, NSCLL, TNBC and small intestine cancer.

In another aspect, also provided herein is use of a pharmaceutical composition, comprising a therapeutically effective amount of a) a LILRB antagonist (e.g., an anti-LILRB2 antibody, an anti-LILRB4 antibody), b) a PD-1/PD-L1 axis inhibitor, or c) both, and one or more pharmaceutically acceptable carriers, in the manufacture of a medicament for treating a disease or condition (e.g., cancer) in a subject in need thereof.

In certain embodiments, the pharmaceutical composition comprising the LILRB antagonist (e.g., an anti-LILRB2 antibody, an anti-LILRB4 antibody) is formulated for use in combination with a PD-1/PD-L1 axis inhibitor.

In another aspect, the pharmaceutical composition is a co-formulation of a therapeutically effective amount of a LILRB antagonist (e.g., an anti-LILRB2 antibody or anti-LILRB4 antibody as defined herein) and a PD-1/PD-L1 axis inhibitor in one or more pharmaceutically acceptable carriers, for treating a disease or condition (e.g., cancer) in a subject in need thereof.

In certain embodiments, the concentration of anti-LILRB2 in formulation for use in combination with a PD-1/PD-L1 axis inhibitor is 50 mg/mL.

In certain embodiments, the concentration of anti-LILRB2 in co-formulation is 50-100 mg/mL.

In certain embodiments, the concentration of anti-LILRB4 in formulation for use in combination with a PD-1/PD-L1 axis inhibitor is 50 mg/mL.

In certain embodiments, the concentration of anti-LILRB4 in co-formulation is 50-100 mg/mL.

### BRIEF DESCRIPTION OF FIGURES

The following drawings form part of the present specification and are included to further demonstrate certain aspects of the present disclosure. The disclosure may be better understood by reference to one or more of these drawings in combination with the detailed description of specific embodiments presented herein.

FIG. 1 is a scheme showing that an antagonist of LILRB works synergistically with a PD-1/PD-L1 axis inhibitor.

FIG. 2 shows that next-generation immunotherapies in combination with standard of care immunotherapies are expected to further increase the survival of cancer patients, as compared to currently available immunotherapies.

FIG. 3A shows anti-LILRB2 phase I dose escalation study design with respect to the DLT (dose-limiting toxicity) window.

FIG. 3B shows anti-LILRB4 phase I dose escalation study design with respect to the DLT window.

FIG. 4A shows anti-LILRB2 solid tumor phase I study design schema.

FIG. 4B shows anti-LILRB4 solid tumor phase 1 study design schema.

FIG. 5 shows anti-LILRB2 serum concentration versus time data at a linear dose range [600 mg (n=8) and 1800 mg (n=4) mono or combo]. Dashed lines are simulated PK curves using the mean PK parameters from 600 mg and 1800 mg individual data analyzed by two compartment model, and triangles are observed data.

FIG. 6 shows anti-LILRB2 serum concentration-receptor occupancy (RO) relationship in blood circulation. RO and anti-LILRB2 serum concentration data from 21 patients were analyzed using an Emax model. The black dots are observed data and smooth line is the fitted curve. Shaded area is the 90% confidence interval (CI) for the fitted curve. 80% and 85% RO (80%-85% RO is considered full saturation using Fluorescence Minus One (FMO) as the background) were projected to achieve at a serum concentration of 25.7 μg/mL and 108 μg/mL, respectively.

FIG. 7A shows sustained receptor occupancy (RO) on blood monocytes during the first dose interval, suggesting full RO at 250 mg of anti-LILRB4 for a few individuals, taking into considerations of patient variabilities.

FIG. 7B shows that LILRB4 expression on blood monocytes at pre- and post-treatment is constant, indicating no internalization post-treatment.

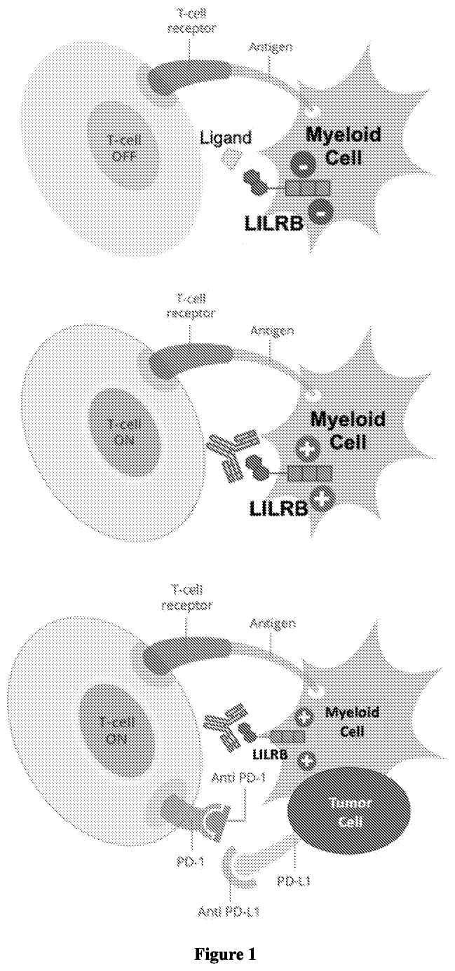

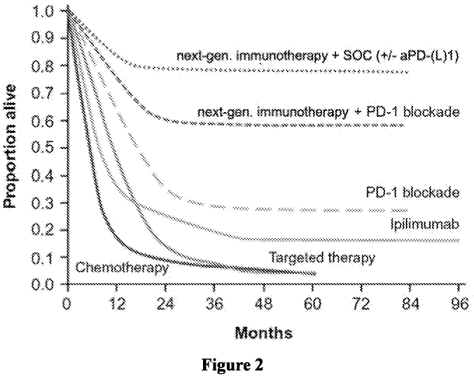

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

As used herein, the term “a,” “an,” “the” and similar terms used in the context of the present disclosure (especially in the context of the claims) are to be construed to cover both the singular and plural unless otherwise indicated herein or clearly contradicted by the context.

The term “LILRB” refers to leukocyte immunoglobulin-like receptor or leukocyte-associated immunoglobulin-like receptor, which is the name of a family of receptors having extracellular immunoglobulin domains. LILRBs are also known as CD85, ILTs, LIR or CD305, and can exert immunomodulatory effects on a wide range of immune cells. The human genes encoding these receptors are found in a gene cluster at chromosomal region 19q13.4, including LILRA1, LILRA2, LILRA3, LILRA4, LILRA5, LILRA6, LILRB1, LILRB2, LILRB3, LILRB4, LILRB5, LILRB6 or LILRA6, LILRB7 or LILRA5, and LAIR1. A subset of LILRs recognize MHC class I molecules (also known as HLA class I in humans).

The term “LILRB2”, used interchangeably with “Leukocyte immunoglobulin-like receptor subfamily B member 2”, “immunoglobulin-like transcript 4 (ILT4) receptor”, or “ILT4”, refers to a protein that in humans is encoded by the LILRB2 gene. LILRB2 belongs to the subfamily B of leukocyte immunoglobulin-like (LIR) receptors that contain two or four extracellular immunoglobulin-like domains, a transmembrane domain, and two to four cytoplasmic immunoreceptor tyrosine-based inhibitory motifs (ITIMs). The receptor is expressed on certain immune cells and hematopoietic progenitor cells, where it binds to MHC class I molecules and to other ligands, thereby transducing a negative signal that inhibits stimulation of an immune response. Binding of LILRB2 to MHC class I also inhibits interaction of MHC class I with CD8, which results in poor T cell priming. It is considered to control inflammatory responses and cytotoxicity to help focus the immune response and limit autoreactivity. Other LILRB2 ligands include angiopoietin-like proteins (ANGPTLs) (e.g., ANGPTL2), semaphorin 4A (SEMA4A), TIM-3, complement split products (CSPs), CD-1 molecules (e.g., CD1d), amyloid-β (AB) oligomer, classical human leukocyte antigen (HLA) class I molecules (HLA-A, HLA-B, HLA-C) and non-classical HLA-class I molecules (HLA-E, HLA-F, HLA-G, and HLA-H).

The term “LILRB4”, used interchangeably with “Leukocyte immunoglobulin-like receptor subfamily B member 4”, “immunoglobulin-like transcript 3 (ILT3) receptor”, “ILT3”, “LIR5” or “CD85K” refers to a protein that in humans is encoded by the LILRB4 gene. LILRB4 belongs to the subfamily B of LIR receptors that contain two or four extracellular immunoglobulin domains, a transmembrane domain, and two to four cytoplasmic immunoreceptor tyrosine-based inhibitory motifs (ITIMs). The receptor is expressed on certain immune cells where, upon ligand (e.g., ApoE, fibronectin) binding, it transduces a negative signal that inhibits stimulation of an immune response. It is thought to control inflammatory responses and cytotoxicity to help focus the immune response and limit autoreactivity.

The term “LAIR1”, used interchangeably with “Leukocyte-associated immunoglobulin-like receptor 1” refers to a protein that in humans is encoded by the LAIR1 gene. LAIR1 has also been designated as CD305 (cluster of differentiation 305) or LAIR-1. LAIR1 is a type I transmembrane glycoprotein that contains one extracellular Ig-like domain and two intracellular ITIMs. Like the genes that encode LILRBs, LAIR1 is localized to the leukocyte receptor complex (LRC) on human chromosome 19q13.4. LAIR1 binds to collagens and other ligands, and its ITIMs recruit SHP-1 and SHP-2. LAIR1 is expressed in T cells, B cells, natural killer (NK) cells, monocytes, macrophages, and dendritic cells, as well as hematopoietic progenitors, such as human CD34+ cells.

As used herein, the term “antagonist” with respect to LILRB refers to any molecule that partially or completely inhibits, blocks, or neutralizes a biological activity of LILRB. Suitable LILRB antagonists may include, without limitation, antibodies, antisense oligonucleotides, peptides, proteins (e.g., fusion proteins), mRNA, siRNA, and small organic molecules. In certain embodiments, the LILRB antagonist is an anti-LILRB antibody.

“Anti-LILRB antibody” as used herein refers to an antibody or antigen-binding fragment thereof that is capable of specific binding to LILRB, such as LILRB2, LILRB4, LILRB5, or LAIR1 with a sufficient affinity, for example, to provide for diagnostic and/or therapeutic use. For example, the term “anti-LILRB2 antibody” as used herein refers to an antibody or antigen-binding fragment thereof that is capable of specific binding to LILRB2 with a sufficient affinity, for example, to provide for diagnostic and/or therapeutic use. For example, the term “anti-LILRB4 antibody” as used herein refers to an antibody or antigen-binding fragment thereof that is capable of specific binding to LILRB4 with a sufficient affinity, for example, to provide for diagnostic and/or therapeutic use.

The term “antibody” as used herein includes any immunoglobulin, monoclonal antibody, polyclonal antibody, multivalent antibody, bivalent antibody, monovalent antibody, multispecific antibody, bispecific antibody, or antibody variant (e.g., affinity variant, glycosylation variant, cysteine-engineered variant, Fc variants (e.g., variants of enhanced or reduced Fcγ receptor IIIA binding affinity), antigen-binding fragments, antibody drug conjugates) that binds to a specific antigen. In certain embodiments, the antibody provided herein refers to an immune cell expressing chimeric antigen receptor, such as CAR-T and CAR-NK.

As used herein, a “bispecific” antibody refers to an artificial antibody which has fragments derived from two different monoclonal antibodies and is capable of binding to two different epitopes. The two epitopes may be present on the same antigen, or they may be present on two different antigens.

The term “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed to form the antibody drug conjugate. Additionally, the antibody drug conjugate can be provided in the form of a fusion protein that may be expressed from a polynucleotide encoding the conjugate.

As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

As used herein, the term “antigen-binding fragment” refers to an antibody fragment formed from a fragment of an antibody comprising one or more CDRs, or any other antibody portion that binds to an antigen but does not comprise an intact native antibody structure. Examples of antigen-binding fragment include, without limitation, a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody. An antigen-binding fragment is capable of binding to the same antigen to which the parent antibody binds. In certain embodiments, an antigen-binding fragment may comprise one or more CDRs from a particular human antibody.

“Fab” with regards to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

“Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

“F(ab′)₂” refers to a dimer of Fab′ that comprises two light chains and part of two heavy chains.

“Fc” with regards to an antibody refers to that portion of the antibody consisting of the second and third constant regions of a first heavy chain bound to the second and third constant regions of a second heavy chain via disulfide bond. IgG and IgM Fc regions contain three heavy chain constant regions (second, third and fourth heavy chain constant regions in each chain). It can be obtained by papain digestion of an antibody. The Fc portion of the antibody is responsible for various effector functions such as ADCC, ADCP and CDC, but does not function in antigen binding.

“Fv” with regards to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

“Single-chain Fv antibody” or “scFv” refers to an engineered antibody consisting of a light chain variable region and a heavy chain variable region connected to one another directly or via a peptide linker sequence (Huston JS et al. Proc Natl Acad Sci USA, 85: 5879 (1988)). A “scFv dimer” refers to a single chain comprising two heavy chain variable regions and two light chain variable regions with a linker. In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, a “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate, V_{H2} and V_{L2} coordinate, and each coordinated pair has a different antigen specificity.

“Single-chain Fv-Fc antibody” or “scFv-Fc” refers to an engineered antibody consisting of a scFv connected to the Fc region of an antibody.

“Camelized single domain antibody,” “heavy chain antibody,” “nanobody” or “HCAb” refers to an antibody that contains two V_{H} domains and no light chains (Riechmann L. and Muyldermans S., J Immunol Methods. December 10; 231 (1-2): 25-38 (1999); Muyldermans S., J Biotechnol. Jun; 74 (4): 277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363 (6428): 446-8 (1993); Nguyen VK. Et al. “Heavy-chain antibodies in Camelidae; a case of evolutionary innovation,” Immunogenetics. Apr; 54 (1): 39-47 (2002); Nguyen VK. Et al. Immunology. May; 109 (1): 93-101 (2003)). The variable domain of a heavy chain antibody (VHH domain) represents the smallest known antigen-binding unit generated by adaptive immune responses (Koch-Nolte F. et al., FASEB J. Nov; 21 (13): 3490-8. Epub 2007 Jun. 15 (2007)). “Diabodies” include small antibody fragments with two antigen-binding sites, wherein the fragments comprise a V_{H} domain connected to a V_{L} domain in a single polypeptide chain (V_{H}-V_{L} Or V_{L}-V_{H}) (see, e.g., Holliger P. et al., Proc Natl Acad Sci USA. July 15; 90 (14): 6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain embodiments, two or more V_{H} domains are covalently joined with a peptide linker to form a bivalent or multivalent domain antibody. The two V_{H} domains of a bivalent domain antibody may target the same or different antigens.

In certain embodiments, a “(dsFv) 2” comprises three peptide chains: two V_{H} moieties linked by a peptide linker and bound by disulfide bridges to two V_{L} moieties.

In certain embodiments, a “bispecific ds diabody” comprises V_{H1}-V_{L2} (linked by a peptide linker) bound to V_{L1}-V_{H2} (also linked by a peptide linker) via a disulfide bridge between V_{H1} and V_{L1}.

In certain embodiments, a “bispecific dsFv” or “dsFv-dsFv” comprises three peptide chains: a V_{H1}-V_{H2} moiety wherein the heavy chains are bound by a peptide linker (e.g., a long flexible linker) and paired via disulfide bridges to V_{L1} and V_{L2} moieties, respectively. Each disulfide paired heavy and light chain has a different antigen specificity.

The term “humanized” as used herein means that the antibody or antigen-binding fragment comprises CDRs derived from non-human animals, FR regions derived from human, and when applicable, constant regions derived from human. In certain embodiments, the amino acid residues of the variable region framework of the humanized LILRB antibody are substituted for sequence optimization. In certain embodiments, the variable region framework sequences of the humanized LILRB antibody chain are at least 65%, 70%, 75%, 80%, 85%, 90%, 95% or 100% identical to the corresponding human variable region framework sequences.

The term “chimeric” as used herein refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

The term “human antibody” as used herein refers to an antibody or antigen-binding fragment that is fully human, e.g., an antibody or antigen-binding fragment that has a heavy chain and/or light chain variable region completely derived from, or synthetized based of, human antibodies.

The term “subject” includes human and non-human animals. Non-human animals include all vertebrates, e.g., mammals and non-mammals, such as non-human primates, mouse, rat, cat, rabbit, sheep, dog, cow, chickens, amphibians, and reptiles. Except when noted, the terms “patient” or “subject” are used herein interchangeably.

The term “diseased tissue” as used herein broadly encompasses diseased cell (such as cancer cell) and tissue (such as tumor tissue).

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

“Cancer” as used herein refers to any medical condition characterized by malignant cell growth or neoplasm, abnormal proliferation, infiltration, or metastasis, and includes both solid tumors and non-solid cancers (e.g., hematologic malignancies) such as leukemias and lymphomas. As used herein, “solid tumor” refers to a solid mass of neoplastic and/or malignant cells and accessory cells (e.g., fibroblasts, immune cells, blood vessel cells).

As used herein, the term “stable disease (SD)”, as defined in the RECIST v1.1, refers to a disease that has stabilized condition, for example, stable tumor size: neither partial response (PR) nor progressive disease (PD) criteria are met. Less than a 20% increase in the sum of target lesion measurements, compared to the smallest sum on study (including baseline). Also, the absolute increase in the sum has to be at least 5 millimeters. Less than a 30% decrease in the sum of diameters of target lesions, compared to the sum at baseline. SD can follow PR only in rare cases, when the sum increases by less than 20% from the nadir, but enough that a previously seen 30% decrease from baseline no longer holds.

As used herein, the term “partial response” (PR), as defined in the RECIST v1.1, refers to at least a 30% decrease in the sum of diameters of target lesions, compared to the sum at baseline.

As used herein, the term “complete response” (CR), as defined in the RECIST v1.1, refers to disappearance of all non-nodal target lesions. Target lymph nodes must reduce to <10 millimeters in the short axis.

The term “immunotherapy” or “immuno-oncology therapies” refers to any therapeutic molecule, RNA, siRNA, mRNA, peptide, antibody, cell or other agent that can modulate a host immune system to generate an immune response to a tumor or cancer in a subject.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

### Methods of Monotherapy

In one aspect, the present disclosure provides a method of treating a disease or condition in a subject in need thereof, comprising administering to the subject a therapeutically effective amount of a LILRB antagonist, such as an anti-LILRB2 antibody (e.g., B2-19-16 as disclosed in PCT/US2021/055927) or anti-LIRB4 antibody (e.g., H7K3m5 as disclosed in PCT/US2021/022029), such that the subject has stable disease, partial response, or complete response for at least 1 month (e.g., 2 months, 3 months, 4 months, 5 months, 6 months, 7 months, 8 months, 9 months, or 10 months).

In certain embodiments, the method comprises administering the LILRB antagonist, such as an anti-LILRB2 antibody (e.g., B2-19-16) or anti-LIRB4 antibody (e.g., H7K3m5), with a dosing interval of once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In certain embodiments, the disease or condition is cancer. In certain embodiments, the cancer is selected from the group consisting of: Merkel cell carcinoma, cholangiocarcinoma, colon neuroendocrine, rectal cancer, colon cancer, pancreatic cancer, HNSCC, breast cancer (e.g., ER+Her2− breast cancer), adrenal carcinoid tumor, bladder cancer, ovarian cancer, and disseminated cancer unknown primary cancer, MSI-H/dMMR CRC, RCC, gastric cancer, NSCLL, TNBC and small intestine cancer. In certain embodiments, the cancer is Merkel cell carcinoma, cholangiocarcinoma, or colon neuroendocrine.

As used herein, the term “therapeutically effective amount”, used interchangeably with the term “therapeutically effective dosage” of a LILRB antagonist used in the methods provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

### Methods of Monotherapy Involving an Anti-LILRB2 Antibody

In certain embodiments, the LILRB antagonist is an anti-LILRB2 antibody (e.g., B2-19-16), which may be administered Q3W at a dosage of about 4000 mg or less, and in certain of these embodiments, the dosage is about 3500 mg or less, about 3000 mg or less, about 2500 mg or less, about 2000 mg or less, about 1800 mg or less, about 1600 mg or less, about 1500 mg or less, about 1200 mg or less, about 1000 mg or less, about 950 mg or less, about 900 mg or less, about 850 mg or less, about 800 mg or less, about 750 mg or less, about 700 mg or less, about 650 mg or less, about 600 mg or less, about 550 mg or less, about 500 mg or less, about 450 mg or less, about 400 mg or less, about 350 mg or less, about 300 mg or less, about 250 mg or less, about 240 mg or less, about 220 mg or less, about 200 mg or less, about 180 mg or less, about 160 mg or less, about 150 mg or less, about 120 mg or less, about 100 mg or less, about 80 mg or less, about 60 mg or less, about 50 mg or less, 40 mg or less, about 30 mg or less, about 20 mg or less, about 10 mg or less, about 8 mg or less, about 6 mg or less, about 4 mg or less, about 2 mg or less, about 1 mg or less, or 0.8 mg or less, or with weekly equivalent amount for any dosing interval from once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In certain embodiments, the LILRB antagonist is an anti-LILRB2 antibody (e.g., B2-19-16), which may be administered Q3W at a therapeutically effective dosage up to about 2000 mg, for example, up to about 1800 mg, up to about 1600 mg, up to about 1400 mg, up to about 1200 mg, up to about 1000 mg, up to about 800 mg, up to about 600 mg, up to about 400 mg, up to about 200 mg, up to about 180 mg, up to about 160 mg, up to about 140 mg, up to about 120 mg, up to about 100 mg, up to about 80 mg, or up to about 60 mg or with weekly equivalent amount for any dosing interval from once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly. It could be understood that weekly equivalent amount could be used for different regimens. For example, the anti-LILRB2 antibody (e.g., B2-19-16) may also be administered at a therapeutically effective dosage up to about 2700 mg Q4W.

In certain embodiments, the anti-LILRB2 antibody (e.g., B2-19-16) may be administered Q3W at a therapeutically effective dosage of about 50 mg to about 2000 mg, for example, about 60 mg to about 1800 mg, about 80 mg to about 1600 mg, about 100 mg to about 1400 mg, about 120 mg to about 1200 mg, about 140 mg to about 1000 mg, about 160 mg to about 800 mg, about 180 mg to about 600 mg, about 200 mg to about 400 mg, or about 300 mg, or with weekly equivalent amount for any dosing interval from once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly. It could be understood that weekly equivalent amount could be used for different regimens.

In certain embodiments, the method comprises administering a LILRB antagonist, such as an anti-LILRB2 antibody (e.g., B2-19-16) at a dose from about 60 mg Q3W to about 1800 mg Q3W (e.g., about 200 mg Q3W to about 1600 mg Q3W, about 400 mg Q3W to about 1200 mg Q3W, about 600 mg Q3W to about 1000 mg Q3W, or about 1200 mg Q3W) to treat a cancer (e.g., rectal cancer, Merkel cell carcinoma, cholangiocarcinoma, pancreatic cancer) in a subject in need thereof.

In certain embodiments, the method comprises administering a LILRB antagonist, such as an anti-LILRB2 antibody (e.g., B2-19-16), at a dose of about 600 mg to 1800 mg Q3W to treat Merkel cell carcinoma in a subject in need thereof.

In certain embodiments, the method comprises administering a LILRB antagonist, such as an anti-LILRB2 antibody (e.g., B2-19-16), at a dose of about 1200 mg Q3W to treat Merkel cell carcinoma in a subject in need thereof.

In certain embodiments, the method comprises administering a LILRB antagonist, such as an anti-LILRB2 antibody (e.g., B2-19-16) at a dose of about 600 mg to 1800 mg Q3W to treat a cancer (e.g., rectal cancer, cholangiocarcinoma, pancreatic cancer) in a subject in need thereof.

In certain embodiments, the method comprises administering a LILRB antagonist, such as an anti-LILRB2 antibody (e.g., B2-19-16) at a dose of about 600 mg Q3W to treat rectal cancer in a subject in need thereof. In certain embodiments, the method comprises administering a LILRB antagonist, such as an anti-LILRB2 antibody (e.g., B2-19-16) at a dose of about 1800 mg Q3W to treat cholangiocarcinoma in a subject in need thereof. In certain embodiments, the method comprises administering a LILRB antagonist, such as an anti-LILRB2 antibody (e.g., B2-19-16) at a dose of about 1800 mg Q3W to treat pancreatic cancer in a subject in need thereof.

In certain embodiments, the subject having the cancer (e.g., rectal cancer, Merkel cell carcinoma, cholangiocarcinoma, pancreatic cancer) has received 2-7 (e.g., 2, 3, 4, 5, 6, 7) prior lines of therapy (e.g., surgical resection, radiotherapy, chemotherapy, immunotherapy, immuno-oncology therapy).

In certain embodiments, the subject has received one or more prior immunotherapy or immuno-oncology therapies, such as pembrolizumab, nivolumab, ipilimumab and/or IL-2/IL-15 agonists.

In certain embodiments, the subject having the cancer (e.g., rectal cancer, Merkel cell carcinoma, cholangiocarcinoma, pancreatic cancer) has received one or more (e.g., 1, 2, 3, or more) lines of PD-1/PD-L1 axis inhibitor treatment (e.g., pembrolizumab, ipilimumab/nivolumab).

In certain embodiments, the subject having the cancer (e.g., rectal cancer, Merkel cell carcinoma, cholangiocarcinoma, pancreatic cancer) has not received PD-1/PD-L1 axis inhibitor treatment.

### Methods of Monotherapy Involving an Anti-LILRB4 Antibody

In certain embodiments, the LILRB antagonist is an anti-LILRB4 antibody (e.g., H7K3m5), which may be administered Q3W at a dosage of about 5 mg to about 4000 mg, about 10 mg to about 3800 mg, about 20 mg to about 3600 mg, about 25 mg to about 3400 mg, about 50 mg to about 3200 mg, about 100 mg to about 3000 mg, about 150 mg to about 2800 mg, about 200 mg to about 2600 mg, about 250 mg to about 2400 mg, about 300 mg to about 2200 mg, about 350 mg to about 2000 mg, about 400 mg to about 1800 mg, about 450 mg to about 1600 mg, about 500 mg to about 1400 mg, about 550 mg to about 1200 mg, about 600 mg to about 1000 mg, about 650 mg to about 800 mg, or about 700 mg, or with weekly equivalent amount for any dosing interval from once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly. In certain embodiments, the anti-LILRB4 antibody is administered at a dosage of about 800 mg Q3W. It could be understood that weekly equivalent amount could be used for different regimens.

In certain embodiments, the method comprises administering an anti-LILRB4 antibody (e.g., H7K3m5) Q3W at a dose from about 250 mg to about 2400 mg, about 300 mg to about 2200 mg, about 350 mg to about 2000 mg, about 400 mg to about 1800 mg, about 450 mg to about 1600 mg, about 500 mg to about 1400 mg, about 550 mg to about 1200 mg, about 600 mg to about 1000 mg, about 650 mg to about 800 mg, or about 700 mg, to treat a cancer (e.g., MSI-H/dMMR CRC, RCC, gastric cancer, NSCLC, TNBC and small intestine cancer) in a subject in need thereof.

It could be understood that weekly equivalent amount could be used for different regimens. For example, in certain embodiments, the method comprises administering an anti-LILRB4 antibody (e.g., H7K3m5) at a dose from about 350 mg to about 3200 mg Q4W, about 550 mg to about 1400 mg Q4W, about 600 mg to about 1200 mg Q4W, about 650 mg to about 1000 mg Q4W, or about 800 mg Q4W.

In certain embodiments, the administration dosage and regimen may change over the course of treatment. For example, in certain embodiments the initial administration dosage may be higher than subsequent administration dosages. In certain embodiments, the administration dosage and regimen may vary over the course of treatment depending on the reaction of the subject.

Dosage regimens may be adjusted to provide the optimum desired response (e.g., a therapeutic response). For example, a single dose may be administered, or several divided doses may be administered over time. In certain embodiments, the LILRB antagonist may be administered once weekly (Q1W), once every two weeks (Q2W), once every three weeks (Q3W), once every four weeks (Q4W), once every five weeks (Q5W), once every six weeks (Q6W), once every seven weeks (Q7W), once every eight weeks (Q8W), once every nine weeks (Q9W), once every ten weeks (Q10W), once every eleven weeks (Q11W), once every twelve weeks (Q12W).

In certain embodiments, the route of administration may be IV or SC.

### Methods of Combination Therapy

In one aspect, the present disclosure provides a method of treating a disease or condition (e.g., cancer) in a subject in need thereof. The method may comprise administering to the subject a therapeutically effective amount of a LILRB antagonist (such as an anti-LILRB2 antibody (e.g., B2-19-16) or anti-LIRB4 antibody (e.g., H7K3m5)) in combination with a therapeutically effective amount of a PD-1/PD-L1 axis inhibitor (e.g., pembrolizumab, cemiplimab, tislelizumab), such that the subject has stable disease, partial response, or complete response for at least 1 month (e.g., 2 months, 3 months, 4 months, 5 months, 6 months, 7 months, 8 months, 9 months, or 10 months).

In certain embodiments, the method comprises administering the LILRB antagonist (such as an anti-LILRB2 antibody (e.g., B2-19-16) or anti-LIRB4 antibody (e.g., H7K3m5) and the PD-1/PD-L1 axis inhibitor (e.g., pembrolizumab, cemiplimab, tislelizumab) with a dosing interval of once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In certain embodiments, the route of administration of the LILRB antagonist (such as an anti-LILRB2 antibody (e.g., B2-19-16) or anti-LIRB4 antibody (e.g., H7K3m5) and/or the PD-1/PD-L1 axis inhibitor (e.g., pembrolizumab, cemiplimab, tislelizumab) may be IV or SC.

In certain embodiments, the subject is determined to have LILRB expression in a diseased tissue or a diseased cell. In certain embodiments, the subject is further determined to have PD-L1 expression in the diseased tissue. In certain embodiments, the subject has been determined to have both LILRB expression and PD-L1 expression in the diseased tissue.

In certain embodiments, the LILRB antagonist and the PD-1/PD-L1 axis inhibitor are respectively administered at a therapeutically effective amount to the subject. As used herein, the term “therapeutically effective amount” of a LILRB antagonist or a PD-1/PD-L1 axis inhibitor used in the methods provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the LILRB antagonist and/or PD-1/PD-L1 axis inhibitor may be administered at a therapeutically effective dosage of about 0.0001 mg/kg to about 100 mg/kg. In certain of these embodiments, LILRB antagonist and/or PD-1/PD-L1 axis inhibitor is administered Q3W at a dosage of about 60 mg/kg or less, and in certain of these embodiments the dosage is 50 mg/kg or less, 25 mg/kg or less, 15 mg/kg or less, 10 mg/kg or less, 5 mg/kg or less, 3 mg/kg or less, 1 mg/kg or less, 0.5 mg/kg or less, or 0.1 mg/kg or less. In some embodiments, the LILRB antagonist and/or PD-1/PD-L1 axis inhibitor is administered Q3W at a dosage of about 4000 mg or less, and in certain of these embodiments, the dosage is about 3500 mg or less, about 3000 mg or less, about 2500 mg or less, about 2000 mg or less, about 1800 mg or less, about 1600 mg or less, about 1500 mg or less, about 1200 mg or less, about 1000 mg or less, about 950 mg or less, about 900 mg or less, about 850 mg or less, about 800 mg or less, about 750 mg or less, about 700 mg or less, about 650 mg or less, about 600 mg or less, about 550 mg or less, about 500 mg or less, about 450 mg or less, about 400 mg or less, about 350 mg or less, about 300 mg or less, about 250 mg or less, about 240 mg or less, about 220 mg or less, about 200 mg or less, about 180 mg or less, about 160 mg or less, about 150 mg or less, about 120 mg or less, about 100 mg or less, about 80 mg or less, about 60 mg or less, about 50 mg or less, 40 mg or less, about 30 mg or less, about 20 mg or less, about 10 mg or less, about 8 mg or less, about 6 mg or less, about 4 mg or less, about 2 mg or less, about 1 mg or less, or 0.8 mg or less, or with weekly equivalent amount for any dosing interval from once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In certain embodiments, the LILRB antagonist may be administered Q3W at a therapeutically effective dosage of about 50 mg to about 2000 mg, for example, about 60 mg to about 1800 mg, about 80 mg to about 1600 mg, about 80 mg to about 1500 mg, about 100 mg to about 1400 mg, about 120 mg to about 1200 mg Q3W, about 140 mg to about 1000 mg, about 140 mg to about 950 mg, about 140 mg to about 900 mg, about 140 mg to about 850 mg, about 160 mg to about 800 mg, about 160 mg to about 750 mg, about 160 mg to about 700 mg, about 160 mg to about 650 mg, about 180 mg to about 600 mg, about 180 mg to about 550 mg, about 180 mg to about 500 mg, about 180 mg to about 450 mg, about 200 mg to about 400 mg, about 200 mg to about 350 mg, or about 250 mg to about 300 mg.

In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

Each therapeutic agent in the combination therapy described herein may be administered simultaneously (e.g., in the same medicament or at the same time), concurrently (i.e., in separate medicaments administered one right after the other in any order or sequentially in any order. Sequential administration may be useful when the therapeutic agents in the combination therapy are in different dosage forms (for example, one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In certain embodiments, the LILRB antagonist may be administered once weekly (Q1W), once every two weeks (Q2W), once every three weeks (Q3W), once every four weeks (Q4W), once every five weeks (Q5W), once every six weeks (Q6W), once every seven weeks (Q7W), once every eight weeks (Q8W), once every nine weeks (Q9W), once every ten weeks (Q10W), once every eleven weeks (Q11W), once every twelve weeks (Q12W). In certain embodiments, the PD-1/PD-L1 axis inhibitor is administered once weekly (Q1W), once every two weeks (Q2W), once every three weeks (Q3W), once every four weeks (Q4W), once every five weeks (Q5W), once every six weeks (Q6W), once every seven weeks (Q7W), once every eight weeks (Q8W), once every nine weeks (Q9W), once every ten weeks (Q10W), once every eleven weeks (Q11W), once every twelve weeks (Q12W).

In certain embodiments, the PD-1/PD-L1 axis inhibitor may be administered once weekly (Q1W), once every two weeks (Q2W), once every three weeks (Q3W), once every four weeks (Q4W), once every five weeks (Q5W), once every six weeks (Q6W), once every seven weeks (Q7W), once every eight weeks (Q8W), once every nine weeks (Q9W), once every ten weeks (Q10W), once every eleven weeks (Q11W), once every twelve weeks (Q12W). In certain embodiments, the PD-1/PD-L1 axis inhibitor is administered once weekly (Q1W), once every two weeks (Q2W), once every three weeks (Q3W), once every four weeks (Q4W), once every five weeks (Q5W), once every six weeks (Q6W), once every seven weeks (Q7W), once every eight weeks (Q8W), once every nine weeks (Q9W), once every ten weeks (Q10W), once every eleven weeks (Q11W), once every twelve weeks (Q12W).

In certain embodiments, the LILRB antagonist and/or PD-1/PD-L1 axis inhibitor are combined or co-formulated in a single dosage form. In certain embodiments, the LILRB antagonist and/or PD-1/PD-L1 axis inhibitor are administered separately. Although the simultaneous administration of the LILRB antagonist and/or PD-1/PD-L1 axis inhibitor may be maintained throughout a period of treatment, anti-cancer activity may also be achieved by subsequent administration of one compound in isolation (for example, the LILRB antagonist following initial combination treatment, or alternatively, PD-1/PD-L1 axis inhibitor following initial combination treatment). In certain embodiments, the PD-1/PD-L1 axis inhibitor is administered after the first 1, 2, 3, 4, 5, 6, 7, 8, 9 or 10 (preferably 2) doses of the LILRB antagonist.

In some embodiments, the LILRB antagonist is administered before administration of the PD-1/PD-L1 axis inhibitor, while in other embodiments, the LILRB antagonist is administered after administration of the PD-1/PD-L1 axis inhibitor. In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration. In one aspect, the present disclosure provides a method of treating a cancer (e.g., cholangiocarcinoma), comprising administering to the subject: (a) a LILRB antagonist, such as an anti-LILRB2 antibody (e.g., B2-19-16) or anti-LILRB4 antibody (e.g., H7K3m5); and (b) PD-1/PD-L1 axis inhibitor, such as pembrolizumab, cemiplimab or tislelizumab, wherein each of the LILRB antagonist and the PD-1/PD-L1 axis inhibitor is administered according to a dosing regimen with a dosing interval of once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly, and wherein the LILRB antagonist is administered (a) in a smaller volume, (b) using a lower concentration, or (c) using a longer dosing interval, or any combination thereof, relative to the volume, concentration or dosing interval that would be required to achieve a desired degree of therapeutic efficacy if the LILRB antagonist was administered as a monotherapy.

In one aspect, the present disclosure provides a method of treating a cancer (e.g., cholangiocarcinoma) in a subject in need thereof, comprising administering to the subject (a) a LILRB antagonist, such as an anti-LILRB2 antibody (e.g., B2-19-16); and (b) PD-1/PD-L1 axis inhibitor, such as pembrolizumab, tislelizumab or cemiplimab. In certain embodiments, the LILRB antagonist is administered Q3W at a dosage of about 4000 mg or less, and in certain of these embodiments, the dosage is about 3500 mg or less, about 3000 mg or less, about 2500 mg or less, about 2000 mg or less, about 1800 mg or less, about 1600 mg or less, about 1500 mg or less, about 1200 mg or less, about 1000 mg or less, about 950 mg or less, about 900 mg or less, about 850 mg or less, about 800 mg or less, about 750 mg or less, about 700 mg or less, about 650 mg or less, about 600 mg or less, about 550 mg or less, about 500 mg or less, about 450 mg or less, about 400 mg or less, about 350 mg or less, about 300 mg or less, about 250 mg or less, about 240 mg or less, about 220 mg or less, about 200 mg or less, about 180 mg or less, about 160 mg or less, about 150 mg or less, about 120 mg or less, about 100 mg or less, about 80 mg or less, about 60 mg or less, about 50 mg or less, 40 mg or less, about 30 mg or less, about 20 mg or less, about 10 mg or less, about 8 mg or less, about 6 mg or less, about 4 mg or less, about 2 mg or less, about 1 mg or less, or 0.8 mg or less, or with weekly equivalent amount for any dosing interval from once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In certain embodiments, the LILRB antagonist is an anti-LILRB2 antibody (e.g., B2-19-16), which is administered Q3W at a dosage of less than about 1800 mg, less than about 1600 mg, less than about 1200 mg, less than about 1000 mg, less than about 800 mg, less than about 600 mg, less than about 400 mg, less than about 200 mg, less than about 100 mg, less than about 80 mg, less than about 60 mg, less than about 40 mg, or less than about 20 mg. In certain embodiments, the LILRB antagonist is administered Q3W at a dosage of about 50 mg to about 2000 mg, for example, about 60 mg to about 1800 mg, about 80 mg to about 1600 mg, about 100 mg to about 1400 mg, about 120 mg to about 1200 mg, about 140 mg to about 1000 mg, about 160 mg to about 800 mg, about 180 mg to about 600 mg, about 200 mg to about 400 mg, or about 300 mg.

In certain embodiments, the a LILRB antagonist is an anti-LILRB4 antibody (e.g., H7K3m5), which is administered Q3W at a dosage of about 5 mg to about 4000 mg, about 10 mg to about 3800 mg, about 20 mg to about 3600 mg, about 25 mg to about 3400 mg, about 50 mg to about 3200 mg, about 100 mg to about 3000 mg, about 150 mg to about 2800 mg, about 200 mg to about 2600 mg, about 250 mg to about 2400 mg, about 300 mg to about 2200 mg, about 350 mg to about 2000 mg, about 400 mg to about 1800 mg, about 450 mg to about 1600 mg, about 500 mg to about 1400 mg, about 550 mg to about 1200 mg, about 600 mg to about 1000 mg, about 650 mg to about 800 mg, or about 700 mg. In certain embodiments, the anti-LILRB4 antibody is administered at a dosage of about 800 mg Q3W.

In certain embodiments, the PD-1/PD-L1 axis inhibitor is administered Q3W at a dosage of about 100 mg to about 500 mg, about 150 mg to about 450 mg, about 200 mg to about 400 mg, about 250 mg to about 350 mg, or about 500 mg. In certain embodiments, the PD-1/PD-L1 axis inhibitor is administered at a dosage of about 200 mg Q3W. In certain embodiments, the PD-1/PD-L1 axis inhibitor is administered at a dosage of about 240 mg Q3W. In certain embodiments, the PD-1/PD-L1 axis inhibitor is administered at a dosage of about 350 mg Q3W. In certain embodiments, the PD-1/PD-L1 axis inhibitor is administered at a dosage of about 360 mg Q3W. In certain embodiments, the PD-1/PD-L1 axis inhibitor is administered at a dosage of about 500 mg Q3W.

In certain embodiments, the PD-1/PD-L1 axis inhibitor is administered Q2W at a dosage of about 100 mg to about 800 mg, about 150 mg to about 500 mg, about 200 mg to about 400 mg, or about 300 mg. In certain embodiments, the PD-1/PD-L1 axis inhibitor is administered at a dosage of about 240 mg Q2W.

In certain embodiments, the PD-1/PD-L1 axis inhibitor is administered Q4W at a dosage of about 100 mg to about 1500 mg, about 150 mg to about 1000 mg, about 200 mg to about 800 mg, or about 500 mg. In certain embodiments, the PD-1/PD-L1 axis inhibitor is administered at a dosage of about 480 mg.

In certain embodiments, the PD-1/PD-L1 axis inhibitor is administered Q6W at a dosage of about 100 mg to about 1500 mg, about 150 mg to about 1000 mg, about 200 mg to about 800 mg, or about 500 mg. In certain embodiments, the PD-1/PD-L1 axis inhibitor is administered at a dosage of about 400 mg Q6W.

The combination therapy of the present disclosure may be used prior to or following surgery to remove a tumor and may be used prior to, during or after radiation therapy. The combination therapy of the present disclosure may be used to treat a tumor that is large enough to be found by palpation or by imaging techniques well known in the art, such as MRI, ultrasound, or CAT scan. In some embodiments, the combination therapy of the present disclosure is used to treat an advanced stage tumor having dimensions of at least about 200 mm³, 300 mm³, 400 mm³, 500 mm³, 750 mm³, or up to 1000 mm³.

In some embodiments, the LILRB antagonist and/or PD-1/PD-L1 axis inhibitor disclosed herein may be administered in combination with one or more additional therapeutic means or agents. For example, the LILRB antagonist and/or PD-1/PD-L1 axis inhibitor disclosed herein may be administered in combination with radiation therapy and/or with another therapeutic agent, for example, another immune activator, an anti-angiogenesis agent, a chemotherapeutic agent, or an anti-cancer drug.

In certain embodiments, the present disclosure provides methods of treating a disease or condition in a subject who has been identified as likely to respond to the combination therapy treatment of LILRB antagonist and PD-1/PD-L1 axis inhibitor. In certain embodiments, the step of treating comprising administering a therapeutically effective amount of LILRB antagonist and a therapeutically effective amount of PD-1/PD-L1 axis inhibitor to the subject.

In certain embodiments, the disease or condition is a cancer selected from the group consisting of: Merkel cell carcinoma, cholangiocarcinoma, colon neuroendocrine, rectal cancer, colon cancer, pancreatic cancer, HNSCC, breast cancer (e.g., ER+Her2− breast cancer), adrenal carcinoid tumor, bladder cancer, ovarian cancer, and disseminated cancer unknown primary cancer. In certain embodiments, the subject has received one or more prior immunotherapy or immuno-oncology therapies, such as an IL-2/IL-15 agonist, a PD-L1XCD27 bispecific antibody, an anti-TIGIT bispecific antibody, and a PD-1/PD-L1 axis inhibitor (e.g., pembrolizumab).

In certain embodiments, the disease or condition is a cancer selected from the group consisting of: MSI-H/dMMR CRC, RCC, gastric cancer, NSCLC, TNBC and small intestine cancer.

### LILRB Antagonist

The LILRB antagonist used in the methods provided herein can comprise an anti-LILRB antibody. The anti-LILRB antibody can be a monoclonal antibody, polyclonal antibody, humanized antibody, chimeric antibody, human antibody, recombinant antibody, bispecific antibody, multi-specific antibody, labeled antibody, bivalent antibody, or anti-idiotypic antibody or antigen-binding fragments thereof. In certain embodiments, the anti-LILRB antagonist is an anti-LILRB CAR-T or CAR-NK cell.

In certain embodiments, the anti-LILRB antibody comprises an anti-LILRB4 antibody, which has been described in PCT/US2016/020838, U.S. 62/368,672, PCT/US2017/044171, U.S. Ser. No. 16/321,745, U.S. Ser. No. 15/696,972, U.S. 62/582,769, U.S. 62/583,825, U.S. 62/584,770, PCT/US2018/059362, U.S. Ser. No. 16/762,273, U.S. 62/730,715, PCT/US2019/050727, U.S. Ser. No. 17/275,838, U.S. Ser. No. 16/678,049, U.S. 62/988,892 and PCT/US2021/022029, disclosure of which have all been incorporated by reference in their entirety.

In certain embodiments, the anti-LILRB antibody comprises an anti-LILRB2 antibody, which has been described in, U.S. 62/970,496, U.S. 63/094,354, U.S. 63/110,317, PCT/US2021/015362, PCT/US2021/055927 and U.S. 63/274,511, disclosure of which have all been incorporated by reference in their entirety. In certain embodiments, the anti-LILRB2 antibody comprises one or more of the following characteristics: a) comprising a constant region of human IgG4 having a mutation of S228P; b) capable of binding to LILRB2 at an EC50 value of less than 2 nM (e.g., about 1.5 nM, about 1.4 nM, about 1.3 nM, about 1.2 nM, about 1.1 nM or about 1.0 nM) as measured by flow cytometry; c) capable of blocking binding of LILRB2 to one or more ligands selected from the group consisting of HLA-G, classical MHC-I, ANGPTLs (e.g., ANGPTL2), CSPs, SEMA4A and CD1d at an IC50 value of less than about 10 nM (e.g., about 8 nM, about 6 nM, about 4 nM, about 2 nM, about 1.5 nM, about 1.4 nM, about 1.3 nM, about 1.2 nM, about 1.1 nM, about 1.0 nM, about 0.8 nM, about 0.6 nM, about 0.4 nM, about 0.2 nM or 0.1 nM) as measured by flow cytometry; and d) having an off-rate after binding to LILRB2 at a koff value of less than 5×10⁻⁴ (e.g., about 4×10⁻⁴, about 3×10⁻⁴)/s.

In certain embodiments, the anti-LILRB2 antibody comprises the same heavy chain complementary determining regions (HCDRs) and the same light chain CDRs (LCDRs) as the HCDRs and the LCDRs of the anti-LILRB2 antibody B2-19-16, wherein the anti-LILRB2 antibody B2-19-16 comprises a heavy chain variable region comprising the amino acid sequence of SEQ ID NO: 7, and a light chain variable region comprising the amino acid sequence of SEQ ID NO: 8.

In certain embodiments, the anti-LILRB2 antibody comprises a heavy chain variable region comprising HCDR1, HCDR2 and HCDR3, and a light chain variable region comprising LCDR1, LCDR2 and LCDR3, wherein the HCDR1 comprises an amino acid sequence of SEQ ID NO: 1, the HCDR2 comprises an amino acid sequence of SEQ ID NO: 2, the HCDR3 comprises an amino acid sequence of SEQ ID NO: 3, the LCDR1 comprises an amino acid sequence of SEQ ID NO: 4, the LCDR2 comprises an amino acid sequence of SEQ ID NO: 5, and the LCDR3 comprises an amino acid sequence of SEQ ID NO: 6.

In certain embodiments, the anti-LILRB2 antibody comprises a heavy chain variable region, and a light chain variable region, wherein the heavy chain variable region comprises an amino acid sequence of SEQ ID NO: 7, and the light chain variable region comprises an amino acid sequence of SEQ ID NO: 8.

In certain embodiments, the anti-LILRB4 antibody is an antibody disclosed in U.S. Pat. No. 10,501,538, US20210371518, or PCT/US2021/022029 as (published WO2021183839).

In certain embodiments, the anti-LILRB4 antibody comprises the same heavy chain complementary determining regions (HCDRs) and the same light chain CDRs (LCDRs) as the HCDRs and the LCDRs of the anti-LILRB2 antibody H7K3m5, wherein the anti-LILRB2 antibody H7K3m5 comprises a heavy chain variable region comprising the amino acid sequence of SEQ ID NO: 15, and a light chain variable region comprising the amino acid sequence of SEQ ID NO: 16.

In certain embodiments, the anti-LILRB4 antibody comprises a heavy chain variable region comprising HCDR1, HCDR2 and HCDR3, and a light chain variable region comprising LCDR1, LCDR2 and LCDR3, wherein the HCDR1 comprises an amino acid sequence of SEQ ID NO: 9, the HCDR2 comprises an amino acid sequence of SEQ ID NO: 10, the HCDR3 comprises an amino acid sequence of SEQ ID NO: 11, the LCDR1 comprises an amino acid sequence of SEQ ID NO: 12, the LCDR2 comprises an amino acid sequence of SEQ ID NO: 13, and the LCDR3 comprises an amino acid sequence of SEQ ID NO: 14.

In certain embodiments, the anti-LILRB4 antibody comprises a heavy chain variable region comprising the amino acid sequence of SEQ ID NO: 15, and a light chain variable region comprising the amino acid sequence of SEQ ID NO: 16.

In certain embodiments, the anti-LILRB antibody comprises an anti-LAIR1 antibody, which has been described in PCT/US2018/012040, U.S. Ser. No. 16/475,223, U.S. 63/057,601, U.S. 63/124,516 and PCT/US2021/043128, disclosure of which have all been incorporated by reference in their entirety.

The LILRB antagonist may increase antigen presentation, reprogram myeloid phenotypes, cause myeloid cells and lymphoid cells to release proinflammatory cytokines, and attract and activate effector cells such as T cells or natural killer (NK) cells etc. in the tumor microenvironment.

### PD-1/PD-L1 Axis Inhibitors

PD-1/PD-L1 axis inhibitors (e.g., PD-1 inhibitor, PD-L1 inhibitor) are a group of immune checkpoint inhibitors that lead to activation, proliferation, cytotoxicity, and/or increase in signaling of T cells and are used as treatment of multiple types of cancers with prominent curative effects.

The term “PD-1/PD-L1 axis inhibitor” is a molecule (e.g., small molecules, antibodies, etc.) that inhibits the interaction between PD-1/PD-L1 axis binding partners, such as PD-1 and PD-L1, to remove the inhibitory effect of T-cell function (e.g., proliferation, cytokine production, and target cell killing) resulting from signaling by the PD-1/PD-L1 signaling axis. The PD-1/PD-L1 axis inhibitor can include a PD-1 inhibitor or PD-L1 inhibitor.

The term “PD-1 inhibitor”, as used herein, refers to a molecule that decreases, abrogates, inhibits, blocks, or interferes with signal transduction resulting from the interaction of PD-1 with one or more of its binding partners, such as PD-L1, PD-L2. In certain embodiments, PD-1 inhibitor is a molecule that blocks the binding of PD-1 to its binding partners, such as PD-L1, PD-L2. For example, a PD-1 inhibitor can be anti-PD-1 antibodies or antigen binding fragments thereof, fusion proteins, oligopeptides, immunoadhesins and other molecules that decrease, abrogate, inhibit, block, or interfere with signal transduction resulting from the interaction of PD-1 with PD-L1 and/or PD-L2. In some embodiments, the PD-1 inhibitor used in the methods provided herein is a bispecific antibody targeting both PD-1 and another molecule, such as PD-L1, PD-L2, CTLA-4, LAG3, TIM-3, Fc receptors, FCRL (1-6), A2AR, CD160, 2B4, TGF-β, TGF-βR, VISTA, BTLA, TIGIT, LAIR1, LILRB1, LILRB2, LILRB3, LILRB4, LILRB5, LILRA (1-6), OX40, CD2, CD27, CD28, CD30, CD40, CD47, SIRPA, CLEC-1, clever-1/stabilin-1, ADGRE, TREM1, TREM2, CD122, ICAM-1, IDO, NKG2D/C, SLAMF7, MS4A4A, SIGLEC (7-15), NKp80, NKG2A, CD160, CD161, CD300, CD163, B7-H3, B7-H4, LFA-1, ICOS, 4-1BB, GITR, BAFFR, HVEM, CD7, LIGHT, TNFR2, TLR (1-9), IL-2, IL-7, IL-15, IL-21, CD16 and CD83. In certain embodiments, the PD-L1 inhibitor used in the methods provided herein is a bispecific antibody targeting both PD-L1 and another molecule, such as TGFβ, 4-1BB, CTLA4, HER2, TIM-3, VEGF, CD47, PD-L1, LAG3, TIGIT, or a LILRB family member.

In some embodiments, the PD-1 inhibitor is an anti-PD-1 antibody. In some embodiments, a PD-1 inhibitor is selected from Table 1.

TABLE 1
Exemplary PD-1 inhibitors

| ID | Drug Name | Company Name |
|---|---|---|
| 1 | Nivolumab (OPDIVO; BMS-936558) | Bristol-Myers Squibb |
| 2 | Dostarlimab (TSR-042) | GSK |
| 3 | Pembrolizumab (KEYTRUDA; MK-3475; | MSD |
| | Lambrolizumab) | |
| 4 | MEDI0680 (AMP-514) | AstraZeneca |
| 5 | Ezabenlimab (BI 754091) | Boehringer Ingelheim |
| 6 | Pidilizumab (CT-011) | CureTech (Medivation owns |
| | | the exclusive right of |
| | | worldwide development) |
| 7 | Cemiplimab (LIBTAYO; REGN2810) | Regeneron; Sanofi Genzyme |
| 8 | Spartalizumab (PDR001) | Novartis |
| 9 | Cetrelimab (JNJ 63723283) | Janssen |
| 10 | Toripalimab (JS001) | Junshi |
| 11 | PF-06801591 | Pfizer |
| 12 | Tislelizumab (BGB-A317) | BeiGene |
| 13 | AMP-224 (GSK-2661380) | AstraZeneca |
| 14 | ABBV-181 | Abbvie |
| 15 | Camrelizumab (SHR-1210) | Hengrui |
| 16 | Sintilimab (Tyvyt; IBI308) | Innovent |
| 17 | Penpulimab (AK105; Aniko; Piamprimab) | Akeso |
| 18 | Zimberelimab | Gloria |
| 19 | Retifanlimab (MGA012) | Incyte, MacroGenics |
| 20 | Serplulimab | Henlius |
| 21 | Balstilimab | Agenus |
| 22 | Geptanolimab | Genor |
| 23 | Prolgolimab | Biocad |
| 24 | Ezabenlimab | Boehringer Ingelheim |
| 25 | Sasanlimab | Pfizer |
| 26 | Pimivalimab | Jounce Therapeutics |
| 27 | Budigalimab | AbbVie |
| 28 | Nofazinlimab | Cstone |
| 29 | Sym021 | Symphogen |
| 30 | ivonescimab (AK112; PD-1/VEGF) | Akeso, Summit |
| 31 | cadonilimab (PD-1/CTLA-4) | Akeso |
| 32 | AK129 (PD-1/LAG-3) | Akeso |

In certain embodiments, a PD-1 inhibitor is any of those under clinical trials before National Medical Products Administration (NMPA), United States Food and Drug Administration (FDA), the European Agency for the Evaluation of Medical Products (EMEA), Japan's Ministry of Health, Labor and Welfare (MHLW), Therapeutic Goods Administration (TGA), Taiwan Food and Drug Administration (TFDA), or their successor(s) in this authority. The detailed information of the PD-1 inhibitors under clinical trials before the above-mentioned authorities can be found in the official websites recording the clinical trial information, such as the China chinadrugtrials database, Chinese Clinical Trial Registry (ChiCTR), the U.S. clinicaltrials database and the European clinicaltrialsregister database.

In certain embodiments, the PD-1 inhibitor is selected from Table 2.

TABLE 2
Other Exemplary PD-1 inhibitors

| | Registration or | | |
|---|---|---|---|
| ID | Acceptance No | Drug Name | Company Name |
| 1 | CXSL2101525 | PD-1 Antibody (PD-1) | EASTERN BIOTECH |
| 2 | CXSL2101383 | ZG005 (PD-1/TIGIT) | ZELGEN |
| 3 | CXSL2101365 | SSGJ-706 Injection (PD-1/X) | GUOJIAN |
| | | | PHARMACEUTICAL |
| 4 | CXSL2101307 | PD-1 Antibody (PD-1) | HEC |
| 5 | CXSL2100075 | LY01015 (PD-1) | BOAN BIOTECH |
| 6 | CXSL2101211 | SSGJ-705 (PD-1/HER2) | GUOJIAN |
| | | | PHARMACEUTICAL |
| 7 | CXSL2101116 | PD1/TIM3 (PD-1/TIM-3) | L&L BIOPHARMA |
| 8 | CXSL2101036 | LBL-015 (PD-1/TGFβ) | LEADS BIOLABS |
| 9 | JXSL2100044 | Balstilimab (PD-1) | BETTA |
| | | | PHARMACEUTICALS |
| 10 | CXSL2100081 | IBI321 (PD-1/TIGIT) | INNOVENT |
| 11 | CXSL2100068 | JS201 (PD-1/TGFß) | TOPALLIANCE |
| 12 | CXSL2100047 | EMB-02 (PD-1/LAG-3) | EPIMAB |
| | | | BIOTHERAPEUTICS |
| 13 | CXSL2000339 | SKB337 (PD-1/CTLA-4) | KELUN |
| | | | PHARMACEUTICAL |
| 14 | JXSL2000214 | Prolgolimab (PD-1) | SHANGHAI PHARMA |
| 15 | CXSL2000353 | Balstilimab (PD-1) | BETTA |
| | | | PHARMACEUTICALS |
| 16 | CXSL2000255 | IBI319 (PD-1/4-1BB) | INNOVENT |
| 17 | CXSL2000144 | PD-1 Monoclonal Antibody | WEIQIDA |
| | | (PD-1) | |
| 18 | CXSL2000111 | AK112 (PD-1/VEGF) | AKESOBIO |
| 19 | CXSL2000042 | BAT1308 (PD-1) | BIO-THERA |
| 20 | CXSL2000032 | Humanized PD-1 Monoclonal | YUNYI |
| | | Antibody (PD-1) | |
| 21 | CXSL2020001 | TY101 (PD-1) | TAYU BIOTECH |
| 22 | CXSL1900132 | MW11 (PD-1) | MABWELL |
| 23 | JXSL 1900119 | INCMGA00012 (PD-1) | ZAI LAB |
| 24 | JXSL1900114 | MGD013 (PD-1/LAG-3) | ZAI LAB |
| 25 | CXSL1900112 | SI-B003 (PD-1/CTLA-4) | BAILI |
| | | | PHARMACEUTICAL |
| 26 | CXSL1900118 | QL1706 (PD-1 + CTLA-4) | QILU |
| | | | PHARMACEUTICAL |
| 27 | CXSL1900098 | HX009 (PD-1/CD47) | HANXBIO |
| 28 | CXSL1900052 | IBI315 (PD-1/HER2) | INNOVENT |
| 29 | CXSL1900059 | Anti-PD-1 (PD-1) | GUOJIAN |
| | | | PHARMACEUTICAL |
| 30 | CXSL1900002 | Anti-PD-1 (PD-1) | ANKEBIO |
| 31 | CXSL1800129 | QL1604 (PD-1) | QILU |
| | | | PHARMACEUTICAL |
| 32 | CXSL1800119 | IBI318 (PD-1/PD-L1) | INNOVENT |
| 33 | CXSL1800084 | PD-1 Antibody (PD-1) | SUMGENBIO/CSPC |
| 34 | CXSL1800058 | RB0004 (PD-1) | REYOUNG |
| 35 | CXSL1800032 | PD-1 Antibody (PD-1) | NEWTIME PHARMACE |
| 36 | CXSL1800030 | STW204 (PD-1) | STAINWEI |
| 37 | CXSL1800027 | CS1003 (PD-1) | CSTONE |
| | | | PHARMACEUTICALS |
| 38 | CXSL1800025 | PD-1 Antibody (PD-1) | SINOCELLTECH |
| 39 | CXSL1700075 | AK105 (PD-1) | AKESOBIO |
| 40 | CXSL1700074 | AK104 (PD-1/CTLA-4) | AKESOBIO |
| 41 | CXSL1700073 | PD-1 Antibody (PD-1) | HENLIUS |
| 42 | CXSL1700012 | CMAB819 (PD-1) | MABTECH |
| 43 | CXSL1600125 | PD-1 Antibody (PD-1) | LIVZON |
| 44 | CXSL1600107 | PD-1 Antibody (PD-1) | LEPU BIOPHARMA |
| 45 | CXSL1600102 | Anti-PD-1 Antibody (PD-1) | BIO-THERA |
| 46 | CXSL1600045 | GLS-010 Injection (PD-1) | GLORIA |
| | | | PHARMACEUTICALS |
| 47 | CXSL1600016 | Geptanolimab Injection (PD-1) | GENOR BIOPHARMA |
| 48 | CXSL1500136 | IBI308 (PD-1) | INNOVENT |
| 49 | CXSL1500096 | BGB-A317 Injection (PD-1) | BEIGENE |
| 50 | CXSL1400138 | PD-1 Antibody (PD-1) | TOPALLIANCE |
| 51 | CXSL1400153 | SHR-1210 for Injection (PD-1) | HENGRUI MEDICINE |
| 52 | CTR20200784 | Recombinant Humanized Anti | ZHUHAI LIVZON |
| | | PD-1 Monoclonal Antibody | MABPHARM INC. |
| | | Injection (LZM009) | |
| 53 | CTR20191862 | Recombinant Humanized Anti | ZHUHAI LIVZON |
| | | PD-1 Monoclonal Antibody | MABPHARM INC. |
| | | Injection | |
| 54 | CTR20180494 | Recombinant Humanized Anti | ZHUHAI LIVZON |
| | | PD-1 Monoclonal Antibody | MABPHARM INC. |
| | | Injection | |
| 55 | CTR20212368 | Recombinant Human Herpes | ZHEJIANG |
| | | Simplex Virus with PD-1 sc-Fv | YANGSHENGTANG |
| | | gene for Injection | BIOTECHNOLOGY CO., |
| | | | LTD./ |
| | | | YANGSHENGTANG |
| | | | CO., LTD./XIAMENT |
| | | | UNIVERSITY |
| 56 | CTR20192099 | Recombinant Human Herpes | ZHEJIANG |
| | | Simplex Virus with Anti PD-1 | YANGSHENGTANG |
| | | Antibody for Injection | BIOTECHNOLOGY CO., |
| | | | LTD./ |
| | | | YANGSHENGTANG |
| | | | CO., LTD./ |
| 57 | CTR20160735 | Recombinant Human Anti-PD-1 | INNOVENT |
| | | Monoclonal Antibody | BIOPHARMACEUTICAL |
| | | | (SUZHOU) CO., LTD. |
| 58 | CTR20201425 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 59 | CTR20201226 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 60 | CTR20200876 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 61 | CTR20200192 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 62 | CTR20200190 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 63 | CTR20192525 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 64 | CTR20192179 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 65 | CTR20191428 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 66 | CTR20190396 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 67 | CTR20190147 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 68 | CTR20182326 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 69 | CTR20182314 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 70 | CTR20181088 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 71 | CTR20180789 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 72 | CTR20180275 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 73 | CTR20180025 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 74 | CTR20171117 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 75 | CTR20170779 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 76 | CTR20170747 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 77 | CTR20170347 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 78 | CTR20170345 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 79 | CTR20170109 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 80 | CTR20160976 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 81 | CTR20160900 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 82 | CTR20160813 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 83 | CTR20160740 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 84 | CTR20160412 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 85 | CTR20160274 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 86 | CTR20160187 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 87 | CTR20160176 | Recombinant Humanized Anti | TAIZHOU JUNSHI |
| | | PD-1 Monoclonal Antibody | BIOMEDICAL |
| | | Injection | TECHNOLOGY CO., |
| | | | LTD./SUZHOU |
| | | | JUNMENG |
| | | | BIOMEDICAL |
| | | | TECHNOLOGY CO., |
| | | | LTD./SHANGHAI |
| | | | JUNSHI BIOSCIENCES |
| | | | CO., LTD. |
| 88 | CTR20201484 | Recombinant Humanized Anti | TAIZHOU HOUDEOKE |
| | | PD-L1 Monoclonal Antibody | TECHNOLOGY CO., |
| | | Injection | LTD. |
| 89 | CTR20201055 | Recombinant Humanized Anti | TAIZHOU HANZHONG |
| | | PD-1 Monoclonal Antibody | BIOMEDICAL CO., |
| | | Injection | LTD./HANGZHOU |
| | | | HANXBIO |
| | | | PHARMACEUTICAL |
| | | | CO., LTD./ |
| | | | ZHONGSHAN AKESO |
| | | | BIOPHARMA, INC./ |
| | | | CHIME BIOLOGICS |
| | | | (WUHAN) CO., LTD. |
| 90 | CTR20202387 | HX008 Injection (Recombinant | TAIZHOU HANZHONG |
| | | Humanized Anti PD-1 | BIOMEDICAL CO., |
| | | Monoclonal Antibody) | LTD./HANGZHOU |
| | | | HANXBIO |
| | | | PHARMACEUTICAL |
| | | | CO., LTD./ |
| | | | ZHONGSHAN AKESO |
| | | | BIOPHARMA, INC. |
| 91 | CTR20202169 | Recombinant Humanized Anti | TAIZHOU HANZHONG |
| | | PD-1 Monoclonal Antibody | BIOMEDICAL CO., |
| | | Injection | LTD./HANGZHOU |
| | | | HANXBIO |
| | | | PHARMACEUTICAL |
| | | | CO., LTD./ |
| | | | ZHONGSHAN AKESO |
| | | | BIOPHARMA, INC. |
| 92 | CTR20202106 | HX008 Injection (Recombinant | TAIZHOU HANZHONG |
| | | Humanized Anti PD-1 | BIOMEDICAL CO., |
| | | Monoclonal Antibody) | LTD./HANGZHOU |
| | | | HANXBIO |
| | | | PHARMACEUTICAL |
| | | | CO., LTD./ |
| | | | ZHONGSHAN AKESO |
| | | | BIOPHARMA, INC. |
| 93 | CTR20202078 | Recombinant Humanized Anti | TAIZHOU HANZHONG |
| | | PD-1 Monoclonal Antibody | BIOMEDICAL CO., |
| | | Injection | LTD./HANGZHOU |
| | | | HANXBIO |
| | | | PHARMACEUTICAL |
| | | | CO., LTD./ |
| | | | ZHONGSHAN AKESO |
| | | | BIOPHARMA, INC. |
| 94 | CTR20191676 | HX008 Injection (Recombinant | TAIZHOU HANZHONG |
| | | Humanized Anti PD-1 | BIOMEDICAL CO., |
| | | Monoclonal Antibody Injection) | LTD./HANGZHOU |
| | | | HANXBIO |
| | | | PHARMACEUTICAL |
| | | | CO., LTD./ |
| | | | ZHONGSHAN AKESO |
| | | | BIOPHARMA, INC. |
| 95 | CTR20181270 | HX008 Injection (Recombinant | TAIZHOU HANZHONG |
| | | Humanized Anti PD-1 | BIOMEDICAL CO., |
| | | Monoclonal Antibody) | LTD./HANGZHOU |
| | | | HANXBIO |
| | | | PHARMACEUTICAL |
| | | | CO., LTD./ |
| | | | ZHONGSHAN AKESO |
| | | | BIOPHARMA, INC. |
| 96 | CTR20180125 | Recombinant Humanized Anti | TAIZHOU HANZHONG |
| | | PD-1 Monoclonal Antibody | BIOMEDICAL CO., |
| | | Injection | LTD./HANGZHOU |
| | | | HANXBIO |
| | | | PHARMACEUTICAL |
| | | | CO., LTD./ |
| | | | ZHONGSHAN AKESO |
| | | | BIOPHARMA, INC. |
| 97 | CTR20210058 | HX008 Injection (Recombinant | TAIZHOU HANZHONG |
| | | Humanized Anti PD-1 | BIOMEDICAL CO., LTD. |
| | | Monoclonal Antibody) | |
| 98 | CTR20212937 | SI-B001 bispecific antibody | SICHUAN BAILI |
| | | injection | PARMACEUTICAL CO., |
| | | | LTD. |
| 99 | CTR20211652 | Recombinant Anti-PD-1 Full | CSPC ZHONGQI |
| | | Human Monoclonal Antibody | PARMACEUTICAL |
| | | Injection | TECHNOLOGY |
| | | | (SHIJIAZHUANG) CO., |
| | | | LTD. |
| 100 | CTR20212885 | Irinotecan Liposome Injection | CSPC OUYI |
| | | | PHARMACEUTICAL |
| | | | CO., LTD. |
| 101 | CTR20201974 | Recombinant Humanized Anti | SINO CELL |
| | | PD-1 Monoclonal Antibody | ENGINEERING CO., |
| | | Injection | LTD. |
| 102 | CTR20200121 | Recombinant Humanized Anti | SINO CELL |
| | | PD-1 Monoclonal Antibody | ENGINEERING CO., |
| | | Injection | LTD. |
| 103 | CTR20192593 | Recombinant Humanized Anti | SINO CELL |
| | | PD-1 Monoclonal Antibody | ENGINEERING CO., |
| | | Injection | LTD. |
| 104 | CTR20191160 | Recombinant Humanized Anti | SINO CELL |
| | | PD-1 Monoclonal Antibody | ENGINEERING CO., |
| | | Injection | LTD. |
| 105 | CTR20191159 | Recombinant Humanized Anti | SINO CELL |
| | | PD-1 Monoclonal Antibody | ENGINEERING CO., |
| | | Injection | LTD. |
| 106 | CTR20182068 | Recombinant Humanized Anti | SINO CELL |
| | | PD-1 Monoclonal Antibody | ENGINEERING CO., |
| | | Injection | LTD. |
| 107 | CTR20202517 | Injection of recombinant | SHANGHAI YUNYI |
| | | human monoclonal antibody | HEALTH |
| | | against PD-1 | TECHNOLOGY |
| | | | DEVELOPMENT CO., |
| | | | LTD. |
| 108 | CTR20211154 | Recombinant Humanized Anti- | SHANGHAI HENLIUS |
| | | PD-1 Monoclonal Antibody | BIOTECH, INC./ |
| | | Injection | SHANGHAI HENLIUS |
| | | | BIOPHARMACEUTICAL |
| | | | CO., LTD. |
| 109 | CTR20200692 | Recombinant Humanized Anti | SHANGHAI HENLIUS |
| | | PD-1 Monoclonal Antibody | BIOTECH, INC./ |
| | | | SHANGHAI HENLIUS |
| | | | BIOPHARMACEUTICAL |
| | | | CO., LTD. |
| 110 | CTR20191104 | Recombinant Humanized Anti | SHANGHAI HENLIUS |
| | | PD-1 Monoclonal Antibody | BIOTECH, INC./ |
| | | Injection | SHANGHAI HENLIUS |
| | | | BIOPHARMACEUTICAL |
| | | | CO., LTD. |
| 111 | CTR20182136 | Recombinant Humanized Anti | SHANGHAI HENLIUS |
| | | PD-1 Monoclonal Antibody | BIOTECH, INC./ |
| | | Injection | SHANGHAI HENLIUS |
| | | | BIOPHARMACEUTICAL |
| | | | CO., LTD. |
| 112 | CTR20210468 | Recombinant Humanized Anti- | SHANGHAI HENLIUS |
| | | PD-1 Monoclonal Antibody | BIOTECH, INC. |
| | | Injection | |
| 113 | CTR20200312 | Recombinant Humanized Anti | SHANGHAI HENLIUS |
| | | PD-1 Monoclonal Antibody | BIOTECH, INC. |
| 114 | CTR20192226 | Recombinant Humanized Anti | SHANGHAI HENLIUS |
| | | PD-1 Monoclonal Antibody | BIOTECH, INC. |
| 115 | CTR20192167 | Recombinant Humanized Anti | SHANGHAI HENLIUS |
| | | PD-1 Monoclonal Antibody | BIOTECH, INC. |
| 116 | CTR20192001 | Recombinant Humanized Anti | SHANGHAI HENLIUS |
| | | PD-1 Monoclonal Antibody | BIOTECH, INC. |
| | | Injection (HLX10) | |
| 117 | CTR20191830 | Recombinant Humanized Anti | SHANGHAI HENLIUS |
| | | PD-1 Monoclonal Antibody | BIOTECH, INC. |
| 118 | CTR20191044 | Recombinant Humanized Anti | SHANGHAI HENLIUS |
| | | PD-1 Monoclonal Antibody | BIOTECH, INC. |
| | | Injection | |
| 119 | CTR20190911 | Recombinant Humanized Anti | SHANGHAI HENLIUS |
| | | PD-1 Monoclonal Antibody | BIOTECH, INC. |
| | | Injection | |
| 120 | CTR20190907 | Recombinant Humanized Anti | SHANGHAI HENLIUS |
| | | PD-1 Monoclonal Antibody | BIOTECH, INC. |
| | | Injection | |
| 121 | CTR20190754 | Recombinant Humanized Anti | SHANGHAI HENLIUS |
| | | PD-1 Monoclonal Antibody | BIOTECH, INC. |
| | | Injection | |
| 122 | CTR20190719 | Recombinant Humanized Anti | SHANGHAI HENLIUS |
| | | PD-1 Monoclonal Antibody | BIOTECH, INC. |
| | | Injection | |
| 123 | CTR20212574 | Recombinant Humanized Anti- | SHANDONG NEWTIME |
| | | PD-1 Monoclonal Antibody | PHARMACEUTICAL |
| | | Injection | CO., LTD. |
| 124 | CTR20212573 | Recombinant Humanized Anti- | SHANDONG NEWTIME |
| | | PD-1 Monoclonal Antibody | PHARMACEUTICAL |
| | | Injection | CO., LTD. |
| 125 | CTR20202433 | Recombinant Humanized Anti- | SHANDONG NEWTIME |
| | | PD-1 Monoclonal Antibody | PHARMACEUTICAL |
| | | Injection | CO., LTD. |
| 126 | CTR20202417 | Recombinant Humanized Anti- | SHANDONG NEWTIME |
| | | PD-1 Monoclonal Antibody | PHARMACEUTICAL |
| | | Injection | CO., LTD. |
| 127 | CTR20201523 | Recombinant Humanized Anti- | SHANDONG NEWTIME |
| | | PD-1 Monoclonal Antibody | PHARMACEUTICAL |
| | | Injection | CO., LTD. |
| 128 | CTR20201338 | Recombinant Humanized Anti- | SHANDONG NEWTIME |
| | | PD-1 Monoclonal Antibody | PHARMACEUTICAL |
| | | Injection | CO., LTD. |
| 129 | CTR20181501 | Recombinant Humanized Anti- | SHANDONG NEWTIME |
| | | PD-1 Monoclonal Antibody | PHARMACEUTICAL |
| | | Injection | CO., LTD. |
| 130 | CTR20212888 | Recombinant Human Anti-PD-1 | SUNSHINE GUOJIAN |
| | | Monoclonal Antibody Injection | PHARMACEUTICAL |
| | | | (SHANGHAI) CO., LTD. |
| 131 | CTR20212425 | Recombinant Human Anti-PD-1 | SUNSHINE GUOJIAN |
| | | Monoclonal Antibody Injection | PHARMACEUTICAL |
| | | | (SHANGHAI) CO., LTD. |
| 132 | CTR20192013 | Recombinant Humanized Anti | SUNSHINE GUOJIAN |
| | | PD-1 Monoclonal Antibody | PHARMACEUTICAL |
| | | Injection | (SHANGHAI) CO., LTD. |
| 133 | CTR20212699 | Recombinant Human Anti-PD-1 | HANGZHOU |
| | | Monoclonal Antibody Injection | SHANGJIAN (SUMGEN) |
| | | | BIOTECHNOLOGY CO., |
| | | | LTD./SHANGJIANMAB |
| | | | (BEIJING) |
| | | | BIOTECHNOLOGY CO., |
| | | | LTD. |
| 134 | CTR20212240 | Recombinant Human Anti-PD-1 | HANGZHOU |
| | | Monoclonal Antibody | SHANGJIAN (SUMGEN) |
| | | | BIOTECHNOLOGY CO., |
| | | | LTD./SHANGJIANMAB |
| | | | (BEIJING) |
| | | | BIOTECHNOLOGY CO., |
| | | | LTD. |
| 135 | CTR20211018 | Recombinant Human Anti-PD-1 | HANGZHOU |
| | | Monoclonal Antibody | SHANGJIAN (SUMGEN) |
| | | | BIOTECHNOLOGY CO., |
| | | | LTD./SHANGJIANMAB |
| | | | (BEIJING) |
| | | | BIOTECHNOLOGY CO., |
| | | | LTD. |
| 136 | CTR20190222 | Recombinant Human Anti-PD-1 | HANGZHOU |
| | | Monoclonal Antibody | SHANGJIAN (SUMGEN) |
| | | | BIOTECHNOLOGY CO., |
| | | | LTD./SHANGJIANMAB |
| | | | (BEIJING) |
| | | | BIOTECHNOLOGY CO., |
| | | | LTD. |
| 137 | CTR20181224 | Recombinant Human Anti-PD-1 | HARBIN GLORIA |
| | | Monoclonal Antibody | PHARMACEUTICALS |
| | | | CO., LTD./WUXI |
| | | | APPTEC CO., LTD. |
| 138 | CTR20170692 | Recombinant Human Anti-PD-1 | HARBIN GLORIA |
| | | Monoclonal Antibody | PHARMACEUTICALS |
| | | | CO., LTD./WUXI |
| | | | APPTEC CO., LTD. |
| 139 | CTR20170433 | Recombinant Human Anti-PD-1 | HARBIN GLORIA |
| | | Monoclonal Antibody | PHARMACEUTICALS |
| | | | CO., LTD./WUXI |
| | | | APPTEC CO., LTD. |
| 140 | CTR20201111 | TY101 Injection | TAYU BIOMEDICAL |
| | | | BEIJING CO., LTD. |
| 141 | CTR20191892 | Recombinant Humanized Anti | ANHUI ANKE |
| | | PD-1 Monoclonal Antibody | BIOTECHNOLOGY |
| | | Injection | (GROUP) CO., LTD. |

The term “PD-L1 inhibitor” is a molecule that decreases, abrogates, inhibits, blocks, or interferes with signal transduction resulting from the interaction of PD-L1 with one or more of its binding partners, such as PD-1, B7-1. For example, the PD-L1 inhibitor can be anti-PD-L1 antibodies or antigen binding fragments thereof, fusion proteins, immunoadhesins, oligopeptides and other molecules that decrease, abrogate, inhibit, block, or interfere with signal transduction resulting from the interaction of PD-L1 with one or more of its binding partners, such as PD-1, B7-1. In some embodiments, the PD-L1 inhibitor used in the methods provided herein is a bispecific antibody targeting both PD-L1 and another molecule, such as PD-1, PD-L2, CTLA-4, LAG3, TIM-3, Fc receptors, FCRL (1-6), A2AR, CD160, 2B4, TGF-β, TGF-βR, VISTA, BTLA, TIGIT, LAIR1, LILRB1, LILRB3, LILRB4, LILRB5, LILRA (1-6), OX40, CD2, CD27, CD28, CD30, CD40, CD47, SIRPA, CLEC-1, clever-1/stabilin-1, ADGRE, TREM1, TREM2, CD122, ICAM-1, IDO, NKG2D/C, SLAMF7, MS4A4A, SIGLEC (7-15), NKp80, NKG2A, CD160, CD161, CD300, CD163, B7-H3, B7-H4, LFA-1, ICOS, 4-1BB, GITR, BAFFR, HVEM, CD7, LIGHT, TNFR2, TLR (1-9), IL-2, IL-7, IL-15, IL-21, CD16 and CD83.

In some embodiments, a PD-L1 inhibitor is an anti-PD-L1 antibody. In some embodiments, the anti-PD-L1 inhibitor is selected from Table 3.

TABLE 3
Exemplary PD-L1 inhibitors

| ID | Drug Name | Company Name |
|---|---|---|
| 1 | Atezolizumab (TECENTRIQ; R05541267; | Genentech |
| | MPDL3280A; RG7446) | |
| 2 | BMS-936559 (BMS 936559; MDX 1105) | Medarex, BMS |
| 3 | Avelumab (BAVENCIO ®, MSB0010718C) | Merck KGaA, Pfizer |
| 4 | lodapolimab (LY3300054) | Eli Lilly and Company |
| 5 | Durvalumab (MEDI4736, IMFINZI ®) | MedImmune, |
| | | AstraZeneca |
| 6 | Pacmilimab (CX-072; Proclaim-CX-072) | CytomX |
| 7 | FAZ053 | Novartis |
| 8 | Envafolimab (KN035) | Alphamab |
| 9 | CS1001 | Cstone |
| 10 | Adebrelimab (SHR-1316) | Atridia; Hengrui |
| 11 | SHR-1701 | Hengrui |
| 12 | Bintrafusp | EMD Serono; Merck |
| | | KGaA |
| 13 | LP002 | Linton |
| 14 | STI-3031 | Sorrento |
| 15 | Cosibelimab | Dana-Farber Cancer |
| | | Institute |
| 16 | NM01 | Lantheus Holdings |
| 17 | LDP | Dragonboat |
| 18 | AMP-224 | Amplimmune |
| 19 | Garivulimab (BGB-A333) | BeiGene |
| 20 | A167, SCD-135 | Kelun |
| 21 | Opucolimab | Henlius |
| 22 | GR1405 | Genrix |

In certain embodiments, a PD-L1 inhibitor is any of those under clinical trials before National Medical Products Administration (NMPA), United States Food and Drug Administration (FDA), the European Agency for the Evaluation of Medical Products (EMEA), Japan's Ministry of Health, Labor and Welfare (MHLW), Therapeutic Goods Administration (TGA), Taiwan Food and Drug Administration (TFDA), or their successor(s) in this authority, particularly preferably the NMPA or its successor(s) in this authority. The detailed information of the PD-L1 inhibitors under clinical trials before the above-mentioned authorities can be found in the official websites recording the clinical trial information, such as the China chinadrugtrials database, Chinese Clinical Trial Registry (ChiCTR), the U.S. clinicaltrials database and the European clinicaltrialsregister database.

In certain embodiments, the PD-L1 inhibitor is selected from Table 4.

TABLE 4
Other Exemplary PD-L1 inhibitors

| | Registration or | | |
|---|---|---|---|
| ID | Acceptance No | Drug Name | Company Name |
| 1 | CXSL2101517 | ATG-101 (PD-L1/4-1BB) | ANTENGENE |
| 2 | CXSL2101471 | HB0028 (PD-L1/TGFβ) | HUAHAI |
| | | | PHARMACEUTICAL |
| 3 | CXSL2101463 | SH006 (PD-L1/TIGIT) | SANHOME |
| 4 | CXSL2101450 | KN052 (PD-L1/OX40) | ALPHAMAB ONCOLOGY |
| 5 | CXSL2101447 | PM1022 (PD-L1/TIGIT) | BIOTHEUS |
| 6 | CXSL2101362 | SH009 Injection (PD- | SANHOME |
| | | L1/CD47) | |
| 7 | CXSL2101346 | TST005 Injection (PD- | TRANSCENTA |
| | | L1/TGFβ) | |
| 8 | CXSL2101351 | LY01019 Injection (PD- | BOAN BIOTECH |
| | | L1/TGFβ) | |
| 9 | CXHL2101540 | AN4005 (PD-L1 Small | ADLAI NORTYE |
| | | Molecule Inhibitor) | |
| 10 | CXHL2101169 | FH-2001 (PD-L1/FGFR | FOSUN PHARMA |
| | | Small Molecule Inhibitor) | |
| 11 | CXSL2101268 | GT90008 (PD-L1/TGFβ) | KINTOR |
| | | | PHARMACEUTICAL |
| 12 | CXSL2101250 | BAT7104 (PD-L1/CD47) | BIO-THERA |
| 13 | CXSL2101194 | LBL-024 (PD-L1/4-1BB) | LEADS BIOLABS |
| 14 | JXSL2101066 | NM21-1480 (PD-L1/4-1BB) | CSTONE |
| | | | PHARMACEUTICALS |
| 15 | CXSL2101175 | PM1003 (PD-L1/4-1BB) | BIOTHEUS |
| 16 | CXSL2101168 | BJ-005 (PD-L1/TGFβ) | BJ BIOSCIENCE |
| 17 | CXSL2101171 | QLF31907 (PD-L1/4-1BB) | QILU PHARMACEUTICAL |
| 18 | CXSL2101058 | 6MW3211 (PD-L1/CD47) | MABWELL |
| 19 | CXSL2101075 | BR102 (PD-L1/TGFβ) | HISUN BIORAY |
| 20 | CXSL2101045 | GNC-035 (PD-L1/CD3/4- | BAILI PHARMACEUTICAL |
| | | 1BB/ROR1) | |
| 21 | CXSL2100073 | Y101D (PD-L1/TGFβ) | YZY BIOPHARMA |
| 22 | CXSL2101000 | SG12473 (PD-L1/CD47) | SUMGEN |
| 23 | CXSL2100007 | IMM2505 (PD-L1/VEGF) | SHENGHE |
| 24 | CXSL2100012 | QLS31901 (PD-L1/TGFβ) | QILU PHARMACEUTICAL |
| 25 | CXHL2000595 | MAX-10181 (PD-L1 Small | MAXINOVEL |
| | | Molecule Inhibitor) | |
| 26 | CXSL2000378 | Q-1802 (PD-L1/Claudin | QUREBIO |
| | | 18.2) | |
| 27 | CXSL2000353 | TQB2858 (PD-L1/TGFβ) | CHIATAI TIANQING |
| 28 | CXSL2000330 | HB0025 (PD-L1/VEGF) | HUAHAI |
| | | | PHARMACEUTICAL |
| 29 | JXSL2000187 | LAE005 (PD-L1) | LAEKNA THERAPEUTICS |
| 30 | CXSL2000290 | IMM2510 (PD-L1/VEGF) | IMMUNEONCO |
| 31 | CXSL2000283 | GNC-039 (PD-L1/4- | BAILI PHARMACEUTICAL |
| | | 1BB/CD3/EGFRvIII) | |
| 32 | CXSL2000242 | IBI323 (PD-L1/LAG-3) | INNOVENT |
| 33 | CXSL2000150 | GNC-038 (PD-L1/4- | BAILI PHARMACEUTICAL |
| | | 1BB/CD3/CD19) | |
| 34 | CXSL1900150 | PM8001 (PD-L1/TGFβ) | BIOTHEUS |
| 35 | CXSL1900125 | IBI322 (PD-L1/CD47) | INNOVENT |
| 36 | CXSL1900120 | ASC22 (PD-L1) | ASCLETIS |
| 37 | CXHL 1900279 | IMMH-010 (PD-L1 Small | CHASE SUN |
| | | Molecule Inhibitor) | |
| 38 | CXSL1900119 | PD-L1 Antibody (PD-L1) | HUAIYU |
| | | | PHARMACEUTICAL |
| 39 | CXSL1900051 | RC98 (PD-L1) | REMEGEN |
| 40 | CXSL1800120 | ES101 (PD-L1/4-1BB) | ELPISCIENCE |
| 41 | CXSL1800119 | IBI318 (PD-1/PD-L1) | INNOVENT |
| 42 | CXSL1800093 | RB0005 (PD-L1) | REYOUNG |
| 43 | CXSL1800090 | PD-L1 Antibody (PD-L1) | DRAGON SAIL/SANJIN |
| 44 | CXSL1800073 | LP002 (PD-L1) | HOUDEOKE/LEPU |
| | | | BIOPHARMA |
| 45 | CXSL1800053 | PD-L1 Antibody (PD-L1) | TOPALLIANCE |
| 46 | CXSL1800050 | KN046 (PD-L1/CTLA-4) | ALPHAMAB ONCOLOGY |
| 47 | CXSL1800028 | PD-L1 Antibody (PD-L1) | HISUN |
| 48 | CXSL1700206 | PD-L1 Antibody (PD-L1) | HENLIUS |
| 49 | CXSL1800010 | SHR-1701 (PD-L1/TGFβ) | HENGRUI MEDICINE |
| 50 | CXSL1700107 | MSB2311 (PD-L1) | MABSPACE BIOSCIENCES |
| 51 | CXSL1700111 | GR1405 (PD-L1) | GENRIXBIO |
| 52 | CXSL1700104 | BGB-A333 (PD-L1) | BEIGENE |
| 53 | CXSL1700004 | ZKAB001 (PD-L1) | LEE'S PHARMACEUTICAL |
| 54 | CXSL1700018 | TQB2450 (PD-L1) | CHIATAI TIANQING |
| 55 | CXSL1700026 | KL-A167 (PD-L1) | KELUN PHARMACEUTICAL |
| 56 | CXSL1700002 | SHR-1316 Injection (PD-L1) | HENGRUI MEDICINE |
| 57 | CXSL1600075 | PD-L1 Antibody (PD-L1) | CSTONE |
| | | | PHARMACEUTICALS |
| 58 | CXSL1600033 | PD-L1 Single Domain | ALPHAMAB ONCOLOGY |
| | | Antibody (PD-L1) | |
| 59 | CTR20213241 | Tislelizumab Injection | BEIGENE (SHANGHAI) |
| | | | BIOTECHNOLOGY CO., |
| | | | LTD. |
| 60 | CTR20212934 | Recombinant Humanized | JIANGSU ALPHAMAB |
| | | PDL1/CTLA-4 Bispecific | BIOPHARMACEUTICALS |
| | | Domain Antibody Fc Fusion | CO., LTD. |
| | | Protein for Injection | |
| 61 | CTR20212416 | Pembrolizumab Injection | MERCK SHARP & DOHME |
| | | | CORP./MSD R&D (CHINA) |
| | | | CO., LTD./MSD IRELAND |
| | | | (CARLOW) |
| 62 | CTR20212163 | Recombinant Human Anti- | HISUN PHARMACEUTICAL |
| | | PD-L1 Monoclonal Antibody | (HANGZHOU) CO., LTD./ |
| | | Injection (HS636) | ZHEJIANG HISUN |
| | | | PHARMACEUTICAL CO., |
| | | | LTD. |
| 63 | CTR20211859 | MK-7684A Injection | MERCK SHARP & DOHME |
| | | | CORP./MSD R&D (CHINA) |
| | | | CO., LTD./MSD IRELAND |
| | | | (CARLOW) |
| 64 | CTR20211776 | Recombinant Anti PD-L1 and | WUHAN YZY BIOPHARMA |
| | | Anti TGF-β Bispecific | CO., LTD. |
| | | Antibody (Y101D) | |
| 65 | CTR20211540 | KN046 (Recombinant | JIANGSU ALPHAMAB |
| | | Humanized PDL1/CTLA-4 | BIOPHARMACEUTICALS |
| | | Bispecific Single Domain | CO., LTD. |
| | | Antibody Fc Fusion Protein | |
| | | Injection) | |
| 66 | CTR20210969 | Recombinant Whole-human | ZHAOKE (GUANGZHOU) |
| | | Anti-PD-L1 Monoclonal | ONCOLOGY DRUG CO., |
| | | Antibody Injection | LTD. |
| | | (ZKAB001) | |
| 67 | CTR20210726 | Recombinant Humanized | JIANGSU ALPHAMAB |
| | | PDL1/CTLA-4 Bispecific | BIOPHARMACEUTICALS |
| | | Domain Antibody Fc Fusion | CO., LTD. |
| | | Protein for Injection | |
| 68 | CTR20210605 | Recombinant Whole-human | ZHAOKE (GUANGZHOU) |
| | | Anti-PD-L1 Monoclonal | ONCOLOGY DRUG CO., |
| | | Antibody Injection | LTD. |
| 69 | CTR20210265 | Recombinant Human Anti- | HISUN PHARMACEUTICAL |
| | | PD-L1 Monoclonal Antibody | (HANGZHOU) CO., LTD./ |
| | | Injection (HS636) | ZHEJIANG HISUN |
| | | | PHARMACEUTICAL CO., |
| | | | LTD. |
| 70 | CTR20210040 | Recombinant Whole-human | ZHAOKE (GUANGZHOU) |
| | | Anti-PD-L1 Monoclonal | ONCOLOGY DRUG CO., |
| | | Antibody Injection | LTD. |
| 71 | CTR20210013 | Recombinant Whole-human | JIANGSU ALPHAMAB |
| | | Anti-PD-L1 Monoclonal | BIOPHARMACEUTICALS |
| | | Antibody Injection | CO., LTD. |
| 72 | CTR20201949 | Human Anti-PD-L1 | SHANGHAI DRAGON SAIL |
| | | Monoclonal Antibody | BIOTECHNOLOGY CO., |
| | | Injection | LTD./ADAGENE |
| | | | PHARMACEUTICAL |
| | | | (SUZHOU) CO., LTD./ |
| | | | DRAGON BOAT |
| | | | BIOPHARMACEUTICAL |
| | | | (SHANGHAI) CO., LTD./ |
| | | | GUILIN SANJIN |
| | | | PHARMACEUTICAL GROUP |
| | | | CO., LTD. |
| 73 | CTR20201901 | Recombinant Humanized | TAIZHOU HOUDEOKE |
| | | Anti-PD-L1 Monoclonal | TECHNOLOGY CO., LTD. |
| | | Antibody Injection (LP002) | |
| 74 | CTR20201699 | Recombinant Humanized | JIANGSU ALPHAMAB |
| | | PDL1/CTLA-4 Bispecific | BIOPHARMACEUTICALS |
| | | Domain Antibody Fc Fusion | CO., LTD. |
| | | Protein for Injection | |
| 75 | CTR20201484 | Recombinant Humanized | TAIZHOU HOUDEOKE |
| | | Anti-PD-L1 Monoclonal | TECHNOLOGY CO., LTD. |
| | | Antibody Injection (LP002) | |
| 76 | CTR20201477 | Recombinant Humanized | TAIZHOU HOUDEOKE |
| | | Anti-PD-L1 Monoclonal | TECHNOLOGY CO., LTD. |
| | | Antibody Injection (LP002) | |
| 77 | CTR20201333 | Recombinant Whole-human | ZHAOKE (GUANGZHOU) |
| | | Anti-PD-L1 Monoclonal | ONCOLOGY DRUG CO., |
| | | Antibody Injection | LTD. |
| 78 | CTR20201294 | Recombinant Humanized | JIANGSU ALPHAMAB |
| | | PDL1/CTLA-4 Bispecific | BIOPHARMACEUTICALS |
| | | Single Domain Antibody Fc | CO., LTD. |
| | | Fusion Protein Injection | |
| 79 | CTR20201006 | Recombinant Humanized | JIANGSU ALPHAMAB |
| | | PDL1/CTLA-4 Bispecific | BIOPHARMACEUTICALS |
| | | Single Domain Antibody Fc | CO., LTD. |
| | | Fusion Protein Injection | |
| 80 | CTR20200638 | Camrelizumab for Injection | SUZHOU SUNCADIA |
| | | | BIOPHARMACEUTICALS |
| | | | CO., LTD./JIANGSU |
| | | | HENGRUI |
| | | | PHARMACEUTICALS CO., |
| | | | LTD./SHANGHAI HENGRUI |
| | | | PHARMACEUTICALS CO., |
| | | | LTD. |
| 81 | CTR20200208 | Recombinant Human Anti- | PHARMACEUTICALS CO., |
| | | PD-L1 Monoclonal Antibody | JIANGSU HUAIYU |
| | | for Injection | LTD. |
| 82 | CTR20200174 | Recombinant Humanized | TAIZHOU JUNSHI |
| | | Anti-PD-L1 Monoclonal | BIOMEDICAL |
| | | Antibody Injection | TECHNOLOGY CO., LTD./ |
| | | | SUZHOU JUNMENG |
| | | | BIOMEDICAL |
| | | | TECHNOLOGY CO., LTD./ |
| | | | SUZHOU UNION |
| | | | BIOPHARMA BIOSCIENCES |
| | | | CO., LTD./SHANGHAI |
| | | | JUNSHI BIOSCIENCES CO., |
| | | | LTD. |
| 83 | CTR20192712 | Recombinant Whole-human | ZHAOKE (GUANGZHOU) |
| | | Anti-PD-L1 Monoclonal | ONCOLOGY DRUG CO., |
| | | Antibody Injection | LTD. |
| 84 | CTR20192678 | Recombinant Whole-human | ZHAOKE (GUANGZHOU) |
| | | Anti-PD-L1 Monoclonal | ONCOLOGY DRUG CO., |
| | | Antibody Injection | LTD. |
| 85 | CTR20192309 | Recombinant Whole-human | ZHAOKE (GUANGZHOU) |
| | | Anti-PD-L1 Monoclonal | ONCOLOGY DRUG CO., |
| | | Antibody Injection | LTD. |
| 86 | CTR20191589 | Pembrolizumab Injection | MSD R&D (CHINA) CO., |
| | | | LTD. |
| 87 | CTR20190693 | Recombinant Human Anti- | SHANGHAI DRAGON SAIL |
| | | PD-L1 Monoclonal Antibody | BIOTECHNOLOGY CO., |
| | | Injection | LTD./ADAGENE |
| | | | PHARMACEUTICAL |
| | | | (SUZHOU) CO., LTD./ |
| | | | DRAGON BOAT |
| | | | BIOPHARMACEUTICAL |
| | | | (SHANGHAI) CO., LTD./ |
| | | | GUILIN SANJIN |
| | | | PHARMACEUTICAL GROUP |
| | | | CO., LTD. |
| 88 | CTR20190423 | Geptanolimab Injection | GENOR BIOPHARMA CO., |
| | | | LTD. |
| 89 | CTR20190190 | Recombinant Human Anti- | HISUN PHARMACEUTICAL |
| | | PD-L1 Monoclonal Antibody | (HANGZHOU) CO., LTD./ |
| | | Injection (HS636) | ZHEJIANG HISUN |
| | | | PHARMACEUTICAL CO., |
| | | | LTD. |
| 90 | CTR20180885 | Recombinant Whole-human | ZHAOKE (GUANGZHOU) |
| | | Anti-PD-L1 Monoclonal | ONCOLOGY DRUG CO., |
| | | Antibody Injection | LTD. |
| 91 | CTR20180882 | Recombinant Whole-human | ZHAOKE (GUANGZHOU) |
| | | Anti-PD-L1 Monoclonal | ONCOLOGY DRUG CO., |
| | | Antibody Injection | LTD. |
| 92 | CTR20180879 | Recombinant Whole-human | ZHAOKE (GUANGZHOU) |
| | | Anti-PD-L1 Monoclonal | ONCOLOGY DRUG CO., |
| | | Antibody Injection | LTD. |
| 93 | CTR20170916 | Recombinant Human Anti- | CSTONE |
| | | PD-L1 Monoclonal Antibody | PHARMACEUTICALS |
| | | Injection (CS1001) | (JIANGSU) CO., LTD./TUO |
| | | | SHI PHARMACEUTICALS |
| | | | (SHANGHAI) CO., LTD. |
| 94 | CTR20160994 | MPDL3280A Injection | F.HOFFMANN-LA ROCHE |
| | | | LTD/GENENTECH INC./ |
| | | | ROCHE (CHINA) |
| | | | INVESTMENT CO., LTD. |
| 95 | CTR20160205 | MK-3475 Injection | MERCK SHARP & DOHME |
| | | | CORP./MSD IRELAND |
| | | | (CARLOW) / MSD R&D |
| | | | (CHINA) CO., LTD. |
| 96 | CTR20160097 | MK-3475 Injection | MERCK SHARP & DOHME |
| | | | CORP./MSD IRELAND |
| | | | (CARLOW)/MSD R&D |
| | | | (CHINA) CO., LTD. |
| 97 | CTR20150883 | Plinabulin Concentrated | DALIAN BEYONDSPRING |
| | | Solution for Injection | PHARMACEUTICALS CO., |
| | | | LTD./BEYONDSPRING |
| | | | PHARMACEUTICALS, INC./ |
| | | | PHARMACEUTICS |
| | | | INTERNATIONAL |
| | | | INCORPORATED (PII) |
| 98 | CTR20212934 | Recombinant Humanized | JIANGSU ALPHAMAB |
| | | PDL1/CTLA-4 Bispecific | BIOPHARMACEUTICALS |
| | | Domain Antibody Fc Fusion | CO., LTD. |
| | | Protein for Injection | |
| 99 | CTR20191219 | Recombinant Humanized | JIANGSU ALPHAMAB |
| | | PDL1/CTLA-4 Bispecific | BIOPHARMACEUTICALS |
| | | Domain Antibody Fc Fusion | CO., LTD. |
| | | Protein for Injection | |
| 100 | CTR20190427 | Recombinant Humanized | JIANGSU ALPHAMAB |
| | | PDL1/CTLA-4 Bispecific | BIOPHARMACEUTICALS |
| | | Domain Antibody Fc Fusion | CO., LTD. |
| | | Protein for Injection | |
| 101 | CTR20190197 | Recombinant Humanized | JIANGSU ALPHAMAB |
| | | PDL1/CTLA-4 Bispecific | BIOPHARMACEUTICALS |
| | | Domain Antibody Fc Fusion | CO., LTD. |
| | | Protein for Injection | |
| 102 | CTR20190195 | Recombinant Humanized | JIANGSU ALPHAMAB |
| | | PDL1/CTLA-4 Bispecific | BIOPHARMACEUTICALS |
| | | Domain Antibody Fc Fusion | CO., LTD. |
| | | Protein for Injection | |
| 103 | CTR20181996 | Recombinant Humanized | JIANGSU ALPHAMAB |
| | | PDL1/CTLA-4 Bispecific | BIOPHARMACEUTICALS |
| | | Domain Antibody Fc Fusion | CO., LTD. |
| | | Protein for Injection | |

In certain embodiments, the PD-1/PD-L1 axis inhibitor used in the methods disclosed herein is a small molecule selected from sulfamonomethoxine, sulfamethizole, the compounds disclosed in Awadasseid et al., Life Sciences (2021) 282:119813, CA-170 developed by Curis and Aurigene, the compounds disclosed in Wu, Q., Jiang, L., Li, Sc. et al., Acta Pharmacol Sin 42, 1-9 (2021) titled “Small molecule inhibitors targeting the PD-1/PD-L1 signaling pathway”, IMMH-101 developed by Hongri Pharma, the compounds disclosed in Zhang et al., European Journal of Medicinal Chemistry: 113356, the compounds disclosed in Zak et al., Oncotarget 2016; 7 (21): 30323-30335 titled “Structural basis for small molecule targeting of the programmed death ligand 1 (PD-L1)”, the compounds disclosed in Lu, CH., Chung, WM., Tsai, C H. et al., Sci Rep 12, 303 (2022) titled “In vitro characterization of a small molecule PD-1 inhibitor that targets the PD-1/PD-L1 interaction”, the compounds disclosed in Song et al., J. Med. Chem. 2021 titled “Design, Synthesis, and Pharmacological Evaluation of Biaryl-Containing PD-1/PD-L1 Interaction Inhibitors Bearing a Unique Difluoromethyleneoxy Linkage”, and the compounds disclosed in Liu et al., Cancer Cell Int 21, 239 (2021) titled “Small molecule inhibitors against PD-1/PD-L1 immune checkpoints and current methodologies for their development: a review”.

The LILRB antagonist and PD-1/PD-L1 axis inhibitor used in the methods provided herein may be administered by any route known in the art, such as for example parenteral (e.g., subcutaneous, intraperitoneal, intravenous, including intravenous infusion, intramuscular, intra-tumoral or intradermal injection) or non-parenteral (e.g., oral, intranasal, intraocular, sublingual, rectal, or topical) routes. In certain embodiments, the administration is via oral, nasal, intravenous, subcutaneous, sublingual, or intramuscular administration.

In certain embodiments, the administration of the composition comprising anti-LILRB antibody is prior to, simultaneously with, or after the administration of the composition comprising PD-1/PD-L1 axis inhibitor.

In certain of these embodiments, a LILRB antagonist that is administered in combination with one or more PD-1/PD-L1 axis inhibitors in the methods provided herein may be administered simultaneously with the one or more PD-1/PD-L1 axis inhibitors, and in certain of these embodiments the LILRB antagonist and the one or more PD-1/PD-L1 axis inhibitors may be administered as part of the same pharmaceutical composition. However, a LILRB antagonist administered “in combination” with a PD-1/PD-L1 axis inhibitor does not have to be administered simultaneously with or in the same composition as the agent. A LILRB antagonist administered prior to or after a PD-1/PD-L1 axis inhibitor is considered to be administered “in combination” with the PD-1/PD-L1 axis inhibitor as the phrase is used herein, even if the LILRB antagonist and the PD-1/PD-L1 axis inhibitor are administered via different routes. Where possible, the PD-1/PD-L1 axis inhibitor administered in combination with the LILRB antagonist are administered according to the schedule listed in the product information sheet of the PD-1/PD-L1 axis inhibitor, or according to the Physicians' Desk Reference 2003 (Physicians' Desk Reference, 57th Ed; Medical Economics Company; ISBN: 1563634457; 57th edition (November 2002)) or protocols well known in the art.

In another aspect, the present disclosure provides use of a pharmaceutical composition, comprising a therapeutically effective amount of a) a LILRB antagonist, b) a PD-1/PD-L1 axis inhibitor, or c) both, and one or more pharmaceutically acceptable carriers, in the manufacture of a medicament for treating a disease or condition (e.g., cancer) in a subject in need thereof. In another aspect, the present disclosure provides use of a pharmaceutical composition comprising a LILRB antagonist in the manufacture of a medicament for treating a disease or condition (e.g., cancer) in a subject in need thereof, wherein the pharmaceutical composition comprising the LILRB antagonist is formulated for use in combination with a PD-1/PD-L1 axis inhibitor.

In some embodiments, the disease or condition is characterized by having: a) LILRB expression in a diseased tissue, and/or b) PD-L1 expression in the diseased tissue or PD-1 expressing immune cells, such as T cells and macrophages.

In certain embodiments, the disease or condition is a cancer selected from the group consisting of Merkel cell carcinoma, colorectal cancer, cholangiocarcinoma, ovarian cancer, pancreatic cancer, head and neck cancer, colon neuroendocrine and disseminated cancer unknown primary cancer, as shown in Table 9. In certain embodiments, the subject has received one or more prior immunotherapy or immuno-oncology therapies, such as an IL-2/IL-15 agonist, a PD-L1×CD27 bispecific antibody, an anti-TIGIT bispecific antibody, and a PD-1/PD-L1 axis inhibitor (e.g., Pembrolizumab).

In certain embodiments, the disease or condition is a cancer selected from the group consisting of: MSI-H/dMMR CRC, RCC, gastric cancer, NSCLL, TNBC and small intestine cancer.

In certain embodiments, the pharmaceutical composition comprises a LILRB antagonist formulated for use in combination with a PD-1/PD-L1 axis inhibitor, wherein both the LILRB antagonist and the PD-1/PD-L1 axis inhibitor selected to co-formulate with each other will each have a fixed dose. In certain embodiments, the unit dosage of the LILRB antagonist and/or the PD-1/PD-L1 axis inhibitor for use in co-formulation is less than about 2400 mg, less than about 2200 mg, less than about 2000 mg, less than about 1800 mg, less than about 1700 mg, less than about 1600 mg, less than about 1500 mg, less than about 1400 mg, less than about 1300 mg, less than about 1200 mg, less than about 1100 mg, less than about 1000 mg, less than about 900 mg, less than about 800 mg, less than about 700 mg, less than about 600 mg, less than about 500 mg, less than about 400 mg, less than about 500 mg, less than about 400 mg, less than about 300 mg, less than about 250 mg, less than about 200 mg, less than about 150 mg, less than about 100 mg, less than about 80 mg, less than about 60 mg, less than about 50 mg, less than about 40 mg, less than about 30 mg, or less than about 20 mg. In certain embodiments, the unit dosage of the LILRB antagonist is about 50 mg to about 2400 mg, for example, about 60 mg to about 2000 mg, 70 mg to about 1800 mg, about 80 mg to about 1600 mg, about 100 mg to about 1400 mg, about 120 mg to about 1200 mg, about 140 mg to about 1000 mg, about 160 mg to about 800 mg, about 180 mg to about 600 mg, about 200 mg to about 400 mg, or about 300 mg

In certain embodiments, the pharmaceutical composition comprises a LILRB antagonist formulated for use in combination with a PD-1/PD-L1 axis inhibitor, wherein the unit dosage of the LILRB antagonist in the pharmaceutical composition is lower than the unit dosage of a LILRB antagonist formulated for use as a monotherapy. In certain embodiments, the unit dosage of the LILRB antagonist for use in combination with a PD-1/PD-L1 axis inhibitor is less than about 2400 mg, less than about 2200 mg, less than about 2000 mg, less than about 1800 mg, less than about 1700 mg, less than about 1600 mg, less than about 1500 mg, less than about 1400 mg, less than about 1300 mg, less than about 1200 mg, less than about 1100 mg, less than about 1000 mg, less than about 900 mg, less than about 800 mg, less than about 700 mg, less than about 600 mg, less than about 500 mg, less than about 400 mg, less than about 500 mg, less than about 400 mg, less than about 300 mg, less than about 250 mg, less than about 200 mg, less than about 150 mg, less than about 100 mg, less than about 80 mg, less than about 60 mg, less than about 50 mg, less than about 40 mg, less than about 30 mg, or less than about 20 mg. In certain embodiments, the unit dosage of the LILRB antagonist is about 50 mg to about 2400 mg, for example, about 60 mg to about 2000 mg, 70 mg to about 1800 mg, about 80 mg to about 1600 mg, about 100 mg to about 1400 mg, about 120 mg to about 1200 mg, about 140 mg to about 1000 mg, about 160 mg to about 800 mg, about 180 mg to about 600 mg, about 200 mg to about 400 mg, or about 300 mg

In certain of these embodiments, the unit dosage of the LILRB antagonist for use in combination with a PD-1/PD-L1 axis inhibitor is about 60 mg/kg or less, and in certain of these embodiments the dosage is 50 mg/kg or less, 25 mg/kg or less, 15 mg/kg or less, 10 mg/kg or less, 5 mg/kg or less, 3 mg/kg or less, 1 mg/kg or less, 0.5 mg/kg or less, or 0.1 mg/kg or less.

In certain embodiments, the LILRB antagonist and a PD-1/PD-L1 axis inhibitor disclosed herein work synergistically to address a wide variety of unmet medical needs. For example, the combination of a LILRB antagonist and a PD-1/PD-L1 axis inhibitor effectively treats cancer types where there are no PD-1/PD-L1 axis inhibitor being approved to treat such cancer types. For another example, the combination of a LILRB antagonist and a PD-1/PD-L1 axis inhibitor effectively treat cancer in naive patients who have had no prior treatment with a PD-1/PD-L1 axis inhibitor, due to the fact that a PD-1/PD-L1 axis inhibitor alone (without the combination with a LILRB antagonist) does not work. In certain embodiments, the combination of a LILRB antagonist and a PD-1/PD-L1 axis inhibitor results in a cancer patient's better response to PD-1/PD-L1 axis inhibitor. In certain embodiments, the combination of a LILRB antagonist and a PD-1/PD-L1 axis inhibitor effectively treats patients who have had prior treatment with a PD-1/PD-L1 axis inhibitor but whose tumors became resistant to prior PD-1/PD-L1 axis inhibitor therapy.

### Kit

In another aspect, the present disclosure provides kits useful in treating a disease or condition (e.g., cancer) in a subject in need thereof, comprising a first container that comprises a LILRB antagonist and optionally a second container that comprises a PD-1/PD-L1 axis inhibitor, and optionally instructions for use of the kit. In some embodiments, the disease or condition is characterized by having: a) LILRB expression in a diseased tissue, and/or b) PD-L1 expression in the diseased tissue or PD-1 expressing immune cells, such as T cells and macrophages. In certain embodiments, the disease or condition is a cancer selected from the group consisting of: Merkel cell carcinoma, cholangiocarcinoma, colon neuroendocrine, rectal cancer, colon cancer, pancreatic cancer, HNSCC, breast cancer (e.g., ER+Her2− breast cancer), adrenal carcinoid tumor, bladder cancer, ovarian cancer, and disseminated cancer unknown primary cancer. In certain embodiments, the cancer is Merkel cell carcinoma, cholangiocarcinoma, or colon neuroendocrine. In certain embodiments, the disease or condition is a cancer selected from the group consisting of: MSI-H/dMMR CRC, RCC, gastric cancer, NSCLL, TNBC and small intestine cancer.

Suitable containers include, for example, vials (e.g., dual chamber vials), bottles, syringes (such as single or dual chamber syringes) and test tubes. The container may be formed from various materials such as glass or plastic. In certain embodiments, the instruction comprises a dosing regimen for administering the LILRB antagonist (a) in a smaller volume, (b) using a lower concentration, or (c) using a longer dosing interval, or any combination thereof, relative to the volume, concentration or dosing interval that would be required to achieve a desired therapeutic efficacy (e.g., stable disease, complete response, or partial response) if the LILRB antagonist was administered as a monotherapy.

In certain embodiments, the instruction comprises a dosing regimen for administering the LILRB antagonist with a dosing interval of Q1W, Q2W, Q3W, Q4W, Q5W, Q6W, Q7W or Q8W. In certain embodiments, the instruction comprises a dosing regimen for administering the LILRB antagonist Q3W with a dosage of less than about 1800 mg, less than about 1600 mg, less than about 1200 mg, less than about 1000 mg, less than about 800 mg, less than about 600 mg, less than about 400 mg, less than about 200 mg, less than about 100 mg, less than about 80 mg, less than about 60 mg, less than about 40 mg, or less than about 20 mg. In certain embodiments, the instruction comprises a dosing regimen for administering the LILRB antagonist at a dosage of about 50 mg to about 2000 mg Q3W, for example, about 60 mg to about 1800 mg Q3W, about 80 mg to about 1600 mg Q3W, about 100 mg to about 1400 mg Q3W, about 120 mg to about 1200 mg Q3W, about 140 mg to about 1000 mg Q3W, about 160 mg to about 800 mg Q3W, about 180 mg to about 600 mg Q3W, about 200 mg to about 400 mg Q3W, or about 300 mg Q3W.

In another aspect, the present disclosure provides kits comprising a LILRB antagonist and a package insert comprising instructions for using the LILRB antagonist in combination with a PD-1/PD-L1 axis inhibitor to treat a disease or condition in a subject in need thereof. In some embodiments, the disease or condition is characterized by having: a) LILRB expression in a diseased tissue, and/or b) PD-L1 expression in the diseased tissue or PD-1 expressing immune cells, such as T cells and macrophages. In certain embodiments, the disease or condition is cancer. In certain embodiments, the cancer is selected from the group consisting of: Merkel cell carcinoma, cholangiocarcinoma, colon neuroendocrine, rectal cancer, colon cancer, pancreatic cancer, HNSCC, breast cancer (e.g., ER+Her2− breast cancer), adrenal carcinoid tumor, bladder cancer, ovarian cancer, and disseminated cancer unknown primary cancer. In certain embodiments, the cancer is Merkel cell carcinoma, cholangiocarcinoma, or colon neuroendocrine. In certain embodiments, the disease or condition is a cancer selected from the group consisting of: MSI-H/dMMR CRC, RCC, gastric cancer, NSCLL, TNBC and small intestine cancer.

In certain embodiments, the instruction comprises a dosing regimen for administering the LILRB antagonist Q3W at a dosage of about 4000 mg or less, and in certain of these embodiments, the dosage is about 3500 mg or less, about 3000 mg or less, about 2500 mg or less, about 2000 mg or less, about 1800 mg or less, about 1600 mg or less, about 1500 mg or less, about 1200 mg or less, about 1000 mg or less, about 950 mg or less, about 900 mg or less, about 850 mg or less, about 800 mg or less, about 750 mg or less, about 700 mg or less, about 650 mg or less, about 600 mg or less, about 550 mg or less, about 500 mg or less, about 450 mg or less, about 400 mg or less, about 350 mg or less, about 300 mg or less, about 250 mg or less, about 240 mg or less, about 220 mg or less, about 200 mg or less, about 180 mg or less, about 160 mg or less, about 150 mg or less, about 120 mg or less, about 100 mg or less, about 80 mg or less, about 60 mg or less, about 50 mg or less, 40 mg or less, about 30 mg or less, about 20 mg or less, about 10 mg or less, about 8 mg or less, about 6 mg or less, about 4 mg or less, about 2 mg or less, about 1 mg or less, or 0.8 mg or less, or with weekly equivalent amount for any dosing interval from once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

In another aspect, the present disclosure provides kits comprising a PD-1/PD-L1 axis inhibitor and a package insert comprising instructions for using the PD-1/PD-L1 axis inhibitor in combination with a LILRB antagonist to treat a disease or condition in a subject in need thereof. In some embodiments, the disease or condition is characterized by having: a) LILRB expression in a diseased tissue or immune cells, and/or b) PD-L1 expression in the diseased tissue or immune cells, or PD-1 expressing immune cells, such as T cells and macrophages.

In another aspect, the present disclosure provides a kit comprising an anti-LILRB antibody and a package insert comprising instructions for using the anti-LILRB antibody in combination with a PD-1/PD-L1 axis inhibitor to treat a disease or condition in a subject having expression of LILRB and/or PD-L1. Any of the PD-1/PD-L1 axis inhibitor described herein or to be shown effective in inhibiting the PD-1/PD-L1 axis signaling may be included in the kit.

As used herein, the term “package insert” refers to instructions included in a commercial package of medicines that contain information about, for example, indications, dosage, usage, administration, contraindications, other medicines to be combined with the packaged product, and/or warnings concerning the use of such medicines.

The kit may further comprise other materials desirable from a commercial and user standpoint, including other buffers, diluents, filters, needles, and syringes.

### Disease or Conditions

The disease or condition to be diagnosed, sensitized, or treated by the methods or kits provided herein can be cancer. In certain embodiments, the cancer cells include but not limited to the cells from the bladder, blood, bone, bone marrow, brain, breast, colon, endometrium, esophagus, gallbladder, gastrointestine, gum, head, kidney, liver, lung, nasopharynx, neck, lip, oral cavity, paranasal sinuses, larynx, ovary, prostate, skin, stomach, pancreas, testis, thyroid, tongue, cervix, or uterus.

In certain embodiments, the cancer is of the histological type selected from the group consisting of: carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain embodiments, the tumor comprises an osteosarcoma, angiosarcoma, rhabdosarcoma, leiomyosarcoma, Ewing sarcoma, glioblastoma, neuroblastoma, or leukemia.

In certain embodiments, the cancer is a solid tumor including adrenal cancer, bile duct carcinoma, bone cancer, brain cancer, breast cancer, cervical cancer, choriocarcinoma, colon cancer, colorectal cancer, esophageal cancer, gastroesophageal junction adenocarcinoma (GEA), eye cancer, gastric cancer, glioblastoma, head and neck cancer, kidney cancer, liver cancer, lung cancer, mesothelioma, melanoma, Merkel cell cancer, nasopharyngeal carcinoma, neuroblastoma, oral cancer, ovarian cancer, pancreatic cancer, penile cancer, pinealoma, prostate cancer, renal cell cancer, retinoblastoma, sarcoma, skin cancer, testicular cancer, thymic carcinoma, thyroid cancer, uterine cancer, and vaginal cancer.

In some embodiments, the cancer is a metastatic, relapsed, refractory or drug-resistant cancer.

In some embodiments, said cancer is a hematologic malignancy, including acute lymphocytic leukemia (ALL), acute myeloid leukemia (AML), B-cell leukemia, blastic plasmacytoid dendritic cell neoplasm (BPDCN), chronic lymphoblastic leukemia (CLL), chronic myelomonocytic leukemia (CMML), chronic myelocytic leukemia (CML), pre-B acute lymphocytic leukemia (Pre-B ALL), diffuse large B-cell lymphoma (DLBCL), extranodal NK/T-cell lymphoma, hairy cell leukemia, HHV8-associated primary effusion lymphoma, plasmablastic lymphoma, primary CNS lymphoma, primary mediastinal large B-cell lymphoma, T-cell/histiocyte-rich B-cell lymphoma, heavy chain disease, Hodgkin's lymphoma, non-Hodgkin's lymphoma, Waldenstrom's macroglobulinemia, multiple myeloma (MM), myelodysplastic syndromes (MDS), myeloproliferative neoplasms, and polycythemia vera.

Examples of cancers applicable to methods of treatment herein include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer (including squamous cell carcinoma of the head and neck).

In certain embodiments, the cancer is selected from the group consisting of gastric cancer, lung cancer (e.g., non-small-cell lung carcinoma (NSCLC)), bronchial cancer, bone cancer, liver and bile duct cancer, cholangiocarcinoma, pancreatic cancer, breast cancer (e.g., ER+HER2− breast cancer, triple-negative breast cancer (TNBC)), liver cancer (e.g., HCC), ovarian cancer, testicle cancer, kidney cancer (e.g., renal cell carcinoma (RCC)), bladder cancer, head and neck cancer (e.g., Head and neck squamous cell carcinoma (HNSCC)), nasopharyngeal cancer, spine cancer, brain cancer, cervix cancer, uterine cancer, endometrial cancer, colon cancer (e.g., MSI-H cancer), colon neuroendocrine, rectal cancer, anal cancer, esophageal cancer (e.g., esophageal squamous cell carcinoma (ESCC)), gastrointestinal cancer, Merkel cell carcinoma, skin cancer, prostate cancer, pituitary cancer, stomach cancer, vagina cancer, thyroid cancer, adrenal carcinoid tumor, glioblastoma, astrocytoma, melanoma, disseminated cancer unknown primary cancer, myelodysplastic syndrome, sarcoma, teratoma, and adenocarcinoma.

In certain embodiments, the cancer is selected from the group consisting of: Merkel cell carcinoma, cholangiocarcinoma, colon neuroendocrine, rectal cancer, colon cancer, pancreatic cancer, HNSCC, breast cancer (e.g., ER+Her2− breast cancer), adrenal carcinoid tumor, bladder cancer, ovarian cancer, and disseminated cancer unknown primary cancer. In certain embodiments, the cancer is Merkel cell carcinoma, cholangiocarcinoma, or colon neuroendocrine. In certain embodiments, the disease or condition is a cancer selected from the group consisting of: MSI-H/dMMR CRC, RCC, gastric cancer, NSCLC, TNBC and small intestine cancer.

TABLE 5
Sequences mentioned or used in the
present application

| SEQ | | |
|---|---|---|
| ID | | |
| NO | Sequence | Annotation |
| 1 | GFTFSNAW | B2-19-16 HCDR1 |
| 2 | IKSKTDTGTT | B2-19-16 HCDR2 |
| 3 | TTDRYSSSWYSPAFDI | B2-19-16 HCDR3 |
| 4 | SSNIGENV | B2-19-16 LCDR1 |
| 5 | YED | B2-19-16 LCDR2 |
| 6 | ATWDESLSGPV | B2-19-16 LCDR3 |
| 7 | EVQLVESGGGLVKPGGSLRLSCAASGFT | B2-19-16 VH |
| | FSNAWMSWVRQAPGKGLEWVGRIKSKTD | |
| | TGTTDYAAPVKGRFTISRDDSKNTLYLQ | |
| | MNSLKTEDTAVYYCTTDRYSSSWYSPAF | |
| | DIWGQGTTVSVSS | |
| 8 | QSVLTQPPSVSEAPRQRVTISCSGSSSNI | B2-19-16 VL |
| | GENVVNWYQQLPGKAPKLLIYYEDLLPSG | |
| | VSDRFSGSKSGTSASLAISGLQSEDEADY | |
| | YCATWDESLSGPVFGGGTKLTVL | |
| 9 | GFSLSSSYWIS | H7K3m5 HCDR1 |
| 10 | WIGSIDSGSVGITYYATWVKG | H7K3m5 HCDR2 |
| 11 | ARHGDNWALDL | H7K3m5 HCDR3 |
| 12 | RASQSISSWLAWY | H7K3m5 LCDR1 |
| 13 | LLIYKASTLAS | H7K3m5 LCDR2 |
| 14 | QHGYIRGDLDNV | H7K3m5 LCDR3 |
| 15 | EVQLVESGGGLVQPGGSLRLSCAASGFS | H7K3m5 VH |
| | LSSSYWISWVRQAPGKGLEWIGSIDSGS | |
| | VGITYYATWVKGRFTISRDNSKNTLYLQ | |
| | MNSLRAEDTAVYYCARHGDNWALDLW | |
| | GQGTLVTVSS | |
| 16 | DIQMTQSPSTLSASVGDRVTITCRASQS | H7K3m5 VL |
| | ISSWLAWYQQKPGKAPKLLIYKASTLAS | |
| | GVPSRFSGSGSGTEFTLTISSLQPDDFA | |
| | TYYCQHGYIRGDLDNVFGGGTKVEIK | |
| 17 | QVQLVQSGVEVKKPGASVKVSCKASGY | Pembrolizumab |
| | TFTNYYMYWVRQAPGQGLEWMGGINP | Heavy Chain |
| | SNGGTNFNEKFKNRVTLTTDSSTTTAYM | |
| | ELKSLQFDDTAVYYCARRDYRFDMGFD | |
| | YWGQGTTVTVSSASTKGPSVFPLAPCSR | |
| | STSESTAALGCLVKDYFPEPVTVSWNSG | |
| | ALTSGVHTFPAVLQSSGLYSLSSVVTVPS | |
| | SSLGTKTYTCNVDHKPSNTKVDKRVESK | |
| | YGPPCPPCPAPEFLGGPSVFLFPPKPKDTL | |
| | MISRTPEVTCVVVDVSQEDPEVQFNWY | |
| | VDGVEVHNAKTKPREEQFNSTYRVVSV | |
| | LTVLHQDWLNGKEYKCKVSNKGLPSSIE | |
| | KTISKAKGQPREPQVYTLPPSQEEMTKN | |
| | QVSLTCLVKGFYPSDIAVEWESNGQPEN | |
| | NYKTTPPVLDSDGSFFLYSRLTVDKSRW | |
| | QEGNVFSCSVMHEALHNHYTQKSLSLSL | |
| | GK | |
| 18 | EIVLTQSPATLSLSPGERATLSCRASKGV | Pembrolizumab |
| | STSGYSYLHWYQQKPGQAPRLLIYLASY | Light Chain |
| | LESGVPARFSGSGSGTDFTLTISSLEPEDF | |
| | AVYYCQHSRDLPLTFGGGTKVEIKRTVA | |
| | APSVFIFPPSDEQLKSGTASVVCLLNNFY | |
| | PREAKVQWKVDNALQSGNSQESVTEQD | |
| | SKDSTYSLSSTLTLSKADYEKHKVYACE | |
| | VTHQGLSSPVTKSFNRGEC | |
| 19 | EVQLLESGGVLVQPGGSLRLSCAASGFT | Cemiplimab Heavy |
| | FSNFGMTWVRQAPGKGLEWVSGISGGG | Chain |
| | RDTYFADSVKGRFTISRDNSKNTLYLQM | |
| | NSLKGEDTAVYYCVKWGNIYFDYWGQ | |
| | GTLVTVSSASTKGPSVFPLAPCSRSTSEST | |
| | AALGCLVKDYFPEPVTVSWNSGALTSG | |
| | VHTFPAVLQSSGLYSLSSVVTVPSSSLGT | |
| | KTYTCNVDHKPSNTKVDKRVESKYGPP | |
| | CPPCPAPEFLGGPSVFLFPPKPKDTLMISR | |
| | TPEVTCVVVDVSQEDPEVQFNWYVDGV | |
| | EVHNAKTKPREEQFNSTYRVVSVLTVLH | |
| | QDWLNGKEYKCKVSNKGLPSSIEKTISK | |
| | AKGQPREPQVYTLPPSQEEMTKNQVSLT | |
| | CLVKGFYPSDIAVEWESNGQPENNYKTT | |
| | PPVLDSDGSFFLYSRLTVDKSRWQEGNV | |
| | FSCSVMHEALHNHYTQKSLSLSLGK | |
| 20 | DIQMTQSPSSLSASVGDSITITCRASLSIN | Cemiplimab Light |
| | TFLNWYQQKPGKAPNLLIYAASSLHGGV | Chain |
| | PSRFSGSGSGTDFTLTIRTLQPEDFATYY | |
| | CQQSSNTPFTFGPGTVVDFRRTVAAPSVF | |
| | IFPPSDEQLKSGTASVVCLLNNFYPREAK | |
| | VQWKVDNALQSGNSQESVTEQDSKDST | |
| | YSLSSTLTLSKADYEKHKVYACEVTHQG | |
| | LSSPVTKSFNRGEC | |
| 21 | QVQLQESGPGLVKPSETLSLTCTVSGFSL | Tislelizumab |
| | TSYGVHWIRQPPGKGLEWIGVIYADGST | Heavy Chain |
| | NYNPSLKSRVTISKDTSKNQVSLKLSSVT | |
| | AADTAVYYCARAYGNYWYIDVWGQGT | |
| | TVTVSSASTKGPSVFPLAPCSRSTSESTA | |
| | ALGCLVKDYFPEPVTVSWNSGALTSGV | |
| | HTFPAVLQSSGLYSLSSVVTVPSSSLGTK | |
| | TYTCNVDHKPSNTKVDKRVESKYGPPCP | |
| | PCPAPPVAGGPSVFLFPPKPKDTLMISRT | |
| | PEVTCVVVAVSQEDPEVQFNWYVDGVE | |
| | VHNAKTKPREEQFNSTYRVVSVLTVVH | |
| | QDWLNGKEYKCKVSNKGLPSSIEKTISK | |
| | AKGQPREPQVYTLPPSQEEMTKNQVSLT | |
| | CLVKGFYPSDIAVEWESNGQPENNYKTT | |
| | PPVLDSDGSFFLYSKLTVDKSRWQEGNV | |
| | FSCSVMHEALHNHYTQKSLSLSLGK | |
| 22 | DIVMTQSPDSLAVSLGERATINCKSSESV | Tislelizumab |
| | SNDVAWYQQKPGQPPKLLINYAFHRFTG | Light |
| | VPDRFSGSGYGTDFTLTISSLQAEDVAVY | Chain |
| | YCHQAYSSPYTFGQGTKLEIKRTVAAPS | |
| | VFIFPPSDEQLKSGTASVVCLLNNFYPRE | |
| | AKVQWKVDNALQSGNSQESVTEQDSKD | |
| | STYSLSSTLTLSKADYEKHKVYACEVTH | |
| | QGLSSPVTKSFNRGEC | |

### Examples

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1: Study Overview for Anti-LILRB2 Monotherapy or its Combination Therapy with a PD-1/PD-L1 Axis Inhibitor

Primary objectives of the study were safety and tolerability and identification of a recommended phase 2 dose (RP2D) for anti-LILRB2 antibody monotherapy or its combination therapy with a PD-1/PD-L1 axis inhibitor in solid tumor patients. Secondary/exploratory objectives include pharmacokinetics (PK) and immunogenicity assessments, antitumor activity as measured by objective response rate (RECIST v1.1), and pharmacodynamic (PD) biomarker effects for anti-LILRB2 antibody monotherapy or its combination therapy with a PD-1/PD-L1 axis inhibitor.

Antibody properties and Mechanism of Action (MOA)

The anti-LILRB2 antibody has fully human IgG4_S228P with specific, high affinity (single-digit nM) binding to LILRB2, blocks binding of LILRB2 to multiple cancer-relevant ligands (HLA-G, ANGPTL2, SEMA4A, CD1d), binds to LILRB2 at an overlapping, but likely distinct epitope from other reference anti-LILRB2 antibodies, and shows a slower off-rate after binding to LILRB2, potentially leading to longer target engagement, better ligand blockade and perhaps better efficacy.

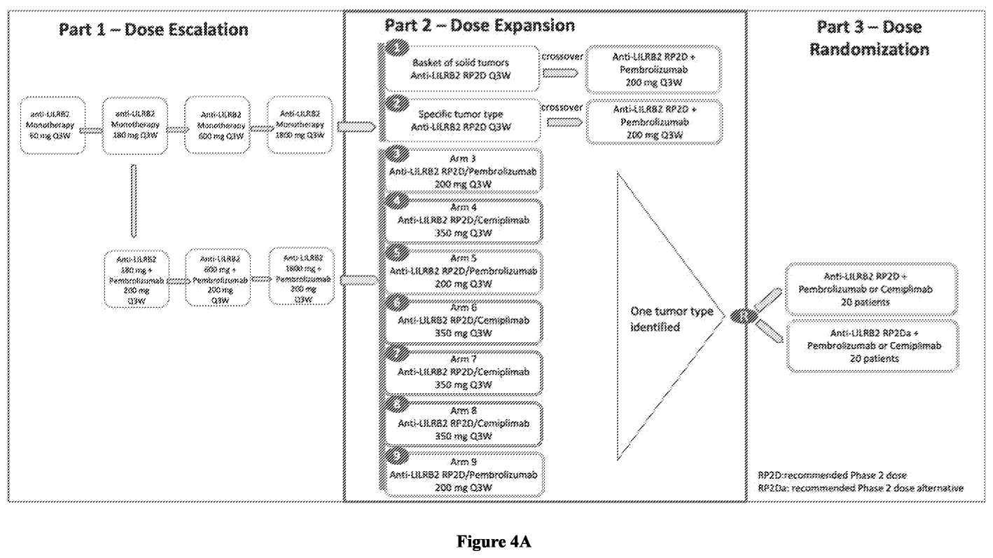

The clinical trial has three parts: Part 1 Dose Escalation, Part 2 Dose Expansion, and Part 3 Dose Randomization (FIG. 4A)

As shown in FIG. 4A, in the Part 1 Dose Escalation, safety and tolerability of varying doses of anti-LILRB2 as monotherapy or in combination with pembrolizumab were studied, in order to determine a RP2D. In Part 2 Dose Expansion, patients with various types of solid tumors were dosed with either anti-LILRB2 alone or in combination with pembrolizumab, cemiplimab or tislelizumab in order to study safety, tolerability and preliminary efficacy of anti-LILRB2 monotherapy and combination with a PD-1 inhibitor. In Part 3, a tumor type that has been studied in the Dose Expansion was selected and patients were randomized into 2 doses of anti-LILRB2 in order to explore safety, toxicity, efficacy relationship with exposure, in order to explore different doses of anti-LILRB2 that is safe and efficacious. Safety, pharmacokinetics (PK), immunogenicity, pharmacodynamics (PD), and efficacy were studied.

Patients recruited for the study have any histologically- or cytologically confirmed advanced/metastatic solid tumor by pathology report and has received, has been intolerant to, or has been ineligible for standard systemic therapy known to confer clinical benefit (e.g., chemotherapy, immunotherapy, radiotherapy).

As shown in Table 6, patients enrolled into anti-LILRB2 clinical trials have median 4.5 prior lines of therapy for monotherapy and 3.6 for combination therapy. These are heavily pre-treated relapsed/refractory solid tumor patients

TABLE 6
Prior Therapies of Solid Tumor Patients enrolled
in Anti-LILRB2 Dose Escalation Clinical Trials

| | Anti-LILRB2 | Anti-LILRB2 + |
|---|---|---|
| | monotherapy | pembrolizumab |
| | treated (n = 12) | combination treated (n = 13) |
| # of prior | average 4.6; | average 3.4; |
| systemic | median 4.5 | median 3.6 |
| therapies | | |
| Prior anti-PD-(L)1 | 4 out of 12 | 6 out of 13 |
| therapy | | |

The study has three arms: 1) treatment of patients with advanced solid tumors with anti-LILRB2 monotherapy; 2) treatment of patients with advanced solid tumors with anti-LILRB2 in combination with a fixed dose of pembrolizumab; and 3) treatment of patients with advanced solid tumors with anti-LILRB2 in combination with a fixed dose of pembrolizumab, cemiplimab or tislelizumab.

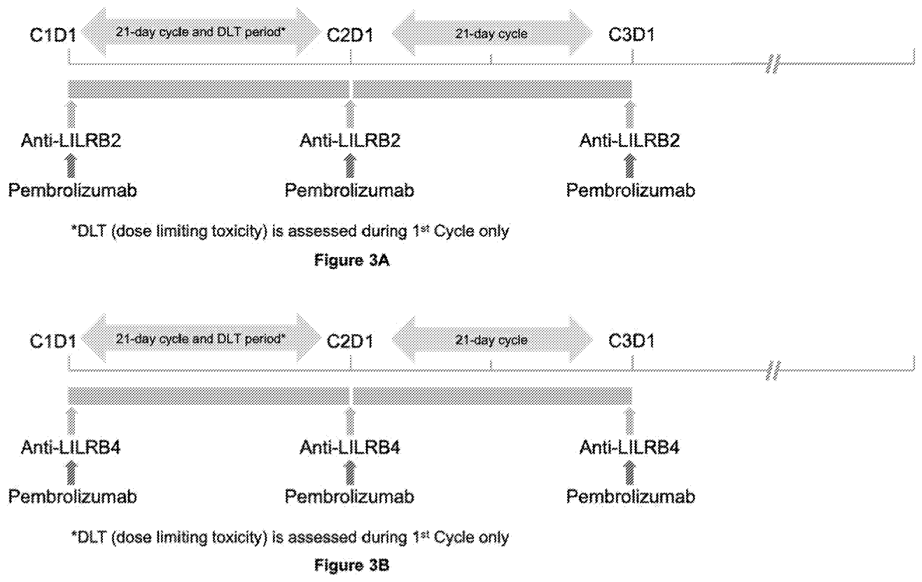

As shown in FIG. 3A, anti-LILRB2 was administered every 21 days (a cycle), i.e., on Cycle 1 Day 1 (C1D1), Cycle 2 Day 1 (C2D1), Cycle 3 Day 1 (C3D1), and so on. Dose limiting toxicity (DLT) was assessed during the first cycle only. Combination therapy initiated after the first two dose levels in monotherapy were cleared. Dose escalation in combination therapy progressed independent of monotherapy dose escalation.

### Example 2: Administration of Anti-LILRB2 or its Combination with a PD-1 Inhibitor is Safe

### 1. Anti-LILRB2 Safety in Dose Escalation

As of 28 Sep. 2022, 28 subjects have been dosed and no serious adverse effects (SAEs) relating to anti-LILRB2 or its combination with a PD-1 inhibitor (e.g., pembrolizumab) were reported. The dosing scheme is shown in Table 7 below. No dose-limiting toxicities (DLTs) were reported. Maximum tolerated dose (MTD) was not reached. Overall, no safety issues were identified in patients subject to the dosing scheme shown in Table 7.

TABLE 7
Dosing Levels

| Dose Group | Dose Level | | # Subjects |
|---|---|---|---|
| Part 1: Anti-LILRB2 | 60 | mg | 2 |
| Monotherapy (n = 12) | 180 | mg | 3 |
| | 600 | mg | 4 |
| | 1800 | mg | 3 |
| Part 1: Anti-LILRB2 + pembrolizumab | 180 mg/200 mg | | 5 |
| Combination (n = 13) | 600 mg/200 mg | | 4 |
| | 1800 mg/200 mg | | 4 |
| Part 2: Anti-LILRB2 Monotherapy (n = 3) | 1200 | mg | 3 |

### 2. 1200 mg is Selected as the RP2D for Part 2 Dose Expansion

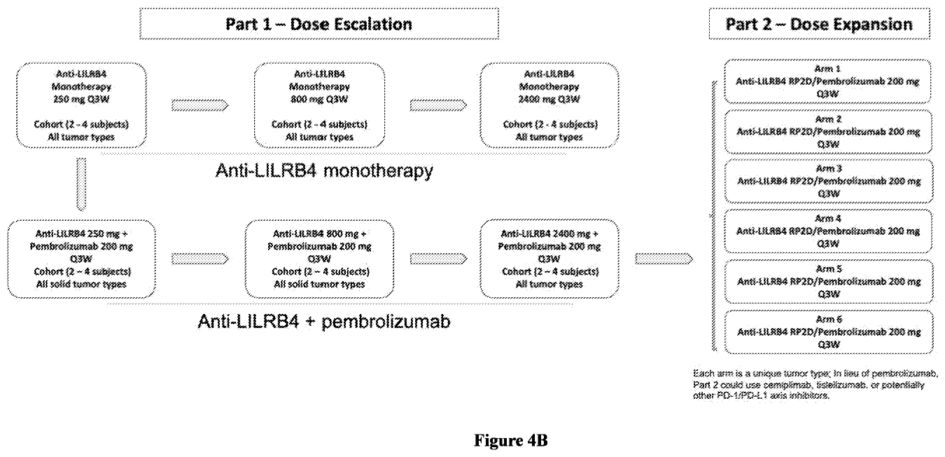

PK and receptor occupancy (RO) data were used to support dose selection due to limited safety and efficacy data available. PK parameters were estimated. PK data at a linear dose range [600 mg (n=8) and 1800 mg (n=4) mono or comb] were analyzed using a two-compartment model (2 CM). As shown in FIG. 5, the serum concentration of anti-LILRB2 at a dose of 1800 mg was higher than that at a dose of 600 mg, while there was no significant difference in the serum concentration of anti-LILRB2 between monotherapy and combination therapy. FIG. 5 also showed that an anti-LILRB2 dosage of 600 mg (mono or combo) resulted in an anti-LILRB2 serum maximum concentration of about 145000 ng/ml upon administration of anti-LILRB2 monotherapy or combination therapy, which decreased over the time, and an anti-LILRB2 dosage of 1800 mg (mono or combo) resulted in an anti-LILRB2 serum maximum concentration of about 809000 ng/mL upon administration of anti-LILRB2 monotherapy or combination therapy, which also decreased over the time.

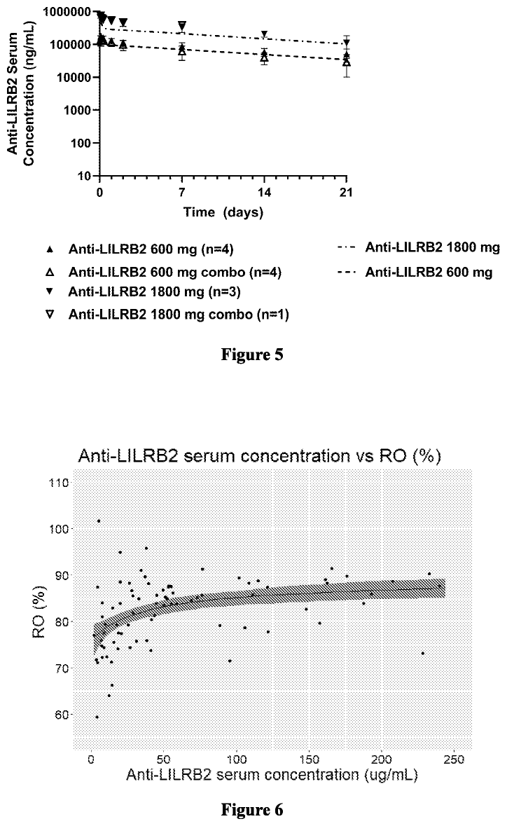

RO and anti-LILRB2 serum concentration data from 21 patients were analyzed using an Emax model. As shown in FIG. 6, the RO increased exponentially as the increase of anti-LILRB2 serum concentration at a lower anti-LILRB2 serum concentration, and then increased slightly as the increase of anti-LILRB2 serum concentration when the anti-LILRB2 serum concentration reached a certain value, and finally reached a plateau when the anti-LILRB2 concentration was around 75000 ng/ml.

Furthermore, PK modeling and PK-RO modeling were integrated to estimate relationship among dose, anti-LILRB2 serum trough concentration, and percentage of RO saturation at pre-dose on Cycle 1 Day 21 (80%-85% is considered as full saturation for current method). As shown in Table 8, full receptor occupancy through 21 days was achieved at a dose of >600 mg, which is consistent with the observed data.

TABLE 8
Project anti-LILRB2 C_{trough} and RO % for a typical
patient on Day 21 following the first dose.

| | Anti-LILRB2 conc. | |
|---|---|---|
| Dose (mg) | (μg/mL) | RO % |
| 180 | 10.3 | 77.4 |
| 200 | 11.4 | 77.7 |
| 300 | 17.2 | 78.7 |
| 400 | 22.9 | 79.6 |
| 500 | 28.6 | 80.4 |
| 600 | 34.3 | 81.0 |
| 700 | 40.1 | 81.6 |
| 800 | 45.8 | 82.1 |
| 900 | 51.5 | 82.5 |
| 1000 | 57.2 | 82.9 |
| 1100 | 63.0 | 83.2 |
| 1200 | 68.7 | 83.5 |
| 1300 | 74.4 | 83.8 |
| 1400 | 80.1 | 84.0 |
| 1500 | 85.8 | 84.3 |
| 1600 | 91.6 | 84.5 |
| 1700 | 97.3 | 84.7 |
| 1800 | 103 | 84.9 |

PK simulation used mean PK parameters with 50% inter-subject variability on clearance (CL) and 45% inter-subject variability on volume of distribution of central compartment (V). Inventors projected that an anti-LILRB2 dosage of 1200 mg could achieved 80% RO saturation (80%-85% is considered as full saturation for current method) in >90% patient population. Inventors also observed that first two patients administered with anti-LILRB2 at a dosage of 1200 mg achieved full RO saturation for the entire dose interval post first dose.

### Example 3: Administration of Anti-LILRB2 or its Combination with a PD-1 Inhibitor is Effective

TABLE 9
Patients with Different Tumor Origins and Best Overall Responses (BOR)
from Dose Escalation Phase by Cohort (n = 23 Efficacy Evaluable).

| Cohort | Tumor Type | Evaluable | BOR | Comments |
|---|---|---|---|---|
| Monotherapy (n = 12) | | | | |
| 60 mg mono | Rectal | Yes | PD | |
| | Colon | Yes | PD | |
| 180 mg mono | Rectal | Yes | PD | |
| | Colon | Yes | PD | |
| | Rectal | Yes | SD | |
| 600 mg mono | Appendix | No | | |
| | carcinoma | | | |
| | Rectal | Yes | SD | |
| | Colon | Yes | PD | |
| | Merkel cell | Yes | CR | |
| | carcinoma | | | |
| 1800 mg mono | Pancreas | Yes | SD | |
| | Breast | Yes | PD | |
| | Cholangiocarcinoma | Yes | SD | SD on monotherapy; |
| | or liver | | | PR after crossover |
| Combo therapy (n = 13) | | | | |
| 180 mg combo | Unknown primary | Yes | SD | |
| | cancer | | | |
| | Cholangiocarcinoma | Yes | PR | PR achieved after |
| | or bile duct | | | anti-LILRB2 dose |
| | | | | increased to 1800 mg |
| | Ovarian | Yes | SD | |
| | Colon | Yes | PD | |
| | Kidney | Yes | PD | |
| 600 mg combo | Pancreas | Yes | SD | |
| | Head and Neck | Yes | SD | |
| | Pancreas | Yes | PD | |
| | Pancreas | Yes | PD | |
| 1800 mg combo | Adrenal | Yes | PD | |
| | Colon | Yes | PR | |
| | neuroendocrine | | | |
| | Bladder | Yes | PD | |
| | Pancreas | No | | |

Table 10 shows patients with complete response (CR), partial response (PR), or stable disease (SD) with anti-LILRB2 monotherapy or in combination with a PD-1/PD-L1 axis inhibitor.

As shown in Table 10, among 23 efficacy evaluable patients, the overall response rate was 9% in monotherapy cohort (1 Merkel cell carcinoma, prior pembrolizumab followed by nivolumab/ipilimumab) and 23% in combination therapy (2 cholangiocarcinoma, 1 MSS CRC with neuroendocrine features). The best overall response was 1 CR, 4 SD among monotherapy, and was 3 PR (including I crossover patient), 4 SD among combination therapy. The 4 responding patients remain on study with an on-going treatment duration of 8 to 12 months as of Feb. 3, 2023.

TABLE 10
Confirmed Response per Investigator-Assessed RECIST v1.1

| | Anti-LILRB2 | | Anti-LILRB2 + | |
|---|---|---|---|---|
| Confirmed | Monotherapy | | pembrolizumab | |
| Response, n (%) | dose escalation n = 12 | | dose escalation n = 13 | |
| Efficacy Evaluable, n | 11 | | 13 | (12 + 1 crossover) |
| ORR | 1 | (9.1) | 3 | (23) |
| CR | 1 | (9.1) | 0 | (0) |
| PR | 0 | (0) | 3 | (23) |
| SD | 4 | (36.4) | 4 | (31) |
| PD | 6 | (54.5) | 6 | (46) |

| | | |
|---|---|---|
| DOR range ^{a}, months | 12+ | 8+-12+ |

^{a} median DOR not reached due to limited sample size and censoring; Data cutoff date: Feb. 3, 2023.

Consistent with the mechanism of action (MOA), clinical benefit correlated with baseline characteristics and post-treatment changes in PD biomarkers including reprogramming of myeloid cells and activation of T cells.

### EXAMPLE 4: STUDY OVERVIEW FOR ANTI-LILRB4 MONOTHERAPY OR ITS Combination Therapy with a PD-1/PD-L1 Axis Inhibitor

The goal of this study is to assess safety and tolerability of increasing doses of anti-LILRB4 either as monotherapy or in combination with pembrolizumab (or cemiplimab, or tislelizumab) in patients with advanced solid tumors and select the recommended Phase 2 dose (RP2D).

This is a Phase 1, open-label, multicenter, dose-escalation and dose-expansion study of anti-LILRB4 in adult subjects with advanced relapsed or refractory solid tumors to study safety, tolerability, pharmacokinetic, pharmacodynamics and clinical activity of anti-LILRB4 as monotherapy or in combination with pembrolizumab (or cemiplimab, or tislelizumab) and to estimate the maximum tolerated dose (MTD) or maximum administered dose (MAD), and to select the RP2D. The study has three arms: 1) treatment with anti-LILRB4 monotherapy, 2) treatment with anti-LILRB4+pembrolizumab (or cemiplimab, or tislelizumab) combination therapy, and 3) treatment with RP2D of anti-LILRB4+pembrolizumab (or cemiplimab, or tislelizumab) combination therapy in solid tumor cohorts (FIG. 4B).

The patients recruited for the dose escalation study must have any histologically or cytologically confirmed advanced or metastatic solid tumor and has received, has been intolerant to, or has been ineligible for standard systemic therapy known to confer clinical benefit. The patients recruited for the dose expansion study must have failed at least one available therapy for the disease under study.

The incidence of treatment-emergent and serious adverse events in patients treated with anti-LILRB4 and anti-LILRB4+pembrolizumab (cemiplimab, or tislelizumab), dose-limiting toxicities (DLTs) with anti-LILRB4 and anti-LILRB4+pembrolizumab (DLTs are measured by the incidence during Cycle 1), the maximum serum concentration (Cmax) of anti-LILRB4, the minimum concentration of anti-LILRB4, the immunogenicity of anti-LILRB4 and anti-LILRB4+pembrolizumab (or cemiplimab, or tislelizumab); anti-tumor activity of anti-LILRB4 and anti-LILRB4+pembrolizumab (or cemiplimab, or tislelizumab), and the receptor occupancy in anti-LILRB4 monotherapy and anti-LILRB4+pembrolizumab (or cemiplimab, or tislelizumab) are measured.

As shown in FIG. 3B, anti-LILRB4 was administered every 21 days (a cycle), i e., on Cycle 1 Day 1 (C1D1), Cycle 2 Day 1 (C2D1), Cycle 3 Day 1 (C3D1), and so on. Dose limiting toxicity (DLT) was assessed during the first cycle only. Combination therapy initiated after the first two dose levels in monotherapy were cleared. Dose escalation in combination therapy progressed independent of monotherapy dose escalation.

### Example 5: Administration of Anti-LILRB4 or its Combination with a PD-1 Inhibitor is Safe

Patients were administered at a dose of 250 mg, 800 mg or 2400 mg and No infusion-related reactions (IRRs), DLTs or other safety issues were identified.

250 mg cohort completed: 4 patients treated. 250 mg+pembrolizumab: 2 patients treated. Opened the 800 mg anti-LILRB4 monotherapy and anti-LILRB4 800 mg+pembrolizumab 200 mg cohorts.

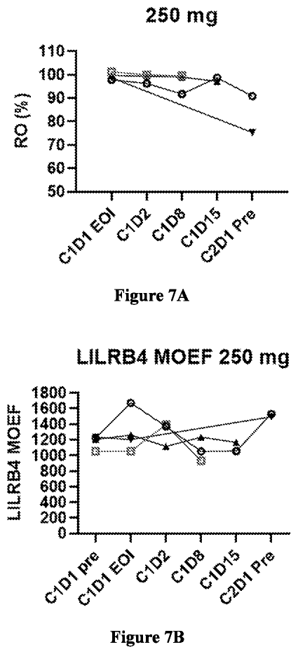

Sustained receptor occupancy (RO) on blood monocytes during the first dose interval, suggesting full RO at 250 mg for a few individuals, taking into considerations of patient variabilities (FIG. 7A). LILRB4 expression at pre- and post-treatment was constant (FIG. 7B), indicating no internalization of LILRB4 post-treatment at a dose of 250 mg.

### Example 6: Administration of Anti-LILRB4 Monotherapy or its Combination with a PD-1 Inhibitor is Effective

The differences between H7K3m5 and a reference anti-LIRB4 antibody 52B8 are summarized in Table 11 below.

TABLE 11
Differential Characteristics of Anti-LILRB4 Antibodies

| | H7K3m5 | 52B8 |
|---|---|---|
| Isotype | Humanized IgG1 | Humanized IgG4 |
| Affinity to human | 0.5 nM | 0.9 nM |
| LILRB4 | | |
| Block APOE | IC₅₀ = 0.18 nM | IC₅₀ = 0.23 nM |
| Block Fibronectin | IC₅₀ = 0.78 nM | No blocking |
| Cross-reactive to non- | yes | yes |
| human primate LILRB4 | | |

Due to the ability of H7K3m5 to block fibronectin binding to LILRB4, it is believed that H7K3m5 may be more effective than the reference antibody 52B8.

TABLE 12
LILRB4 Copy Number per Cell at Baseline
LILRB4 copy number per cell at baseline

| Cell type | MSS CRC | Pancreatic | Gastric | Small Intestine |
|---|---|---|---|---|
| pDC | 13654 | 18676 | 18193 | 14341 |
| Monocyte | 10290 | 10395 | 9997 | 8927 |
| M-MDSC | 8815 | 5560 | 8961 | 3559 |
| mDC | 1861 | 2250 | 1728 | 2183 |

1. A method of treating a disease or condition in a subject in need thereof, comprising:
administering to the subject a therapeutically effective amount of an antagonist of LILRB, such that the subject achieves clinical or objective response, such as stable disease, partial response, or complete response for at least 1 month (e.g., 2 months, 3 months, 4 months, 5 months, 6 months, 7 months, 8 months, 9 months, or 10 months).

2. The method of claim 1, further comprising administering to the subject a therapeutically effective amount of a PD-1/PD-L1 axis inhibitor.

3. The method of claim 1 or 2, wherein the antagonist of LILRB is an anti-LILRB1 antibody, an anti-LILRB2 antibody, an anti-LILRB3 antibody, an anti-LILRB4 antibody, an anti-LILRB5 antibody or an anti-LAIR1 antibody.

4. The method of claim 1 or 2, comprising administering the antagonist of LILRB and optionally the PD-1/PD-L1 axis inhibitor with a dosing interval of once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly, intravenously (IV) or subcutaneously (SC).

5. The method of claim 3, wherein the anti-LILRB2 antibody:
a) comprises a constant region of human IgG4 having a mutation of S228P;
b) is capable of specifically binding to LILRB2 at an EC50 value of less than 2 nM (e.g., about 1.5 nM, about 1.4 nM, about 1.3 nM, about 1.2 nM, about 1.1 nM or about 1.0 nM) as measured by flow cytometry, ELISA, or reporter gene expression;
c) is capable of blocking binding of LILRB2 to one or more ligands selected from the group consisting of HLA-G, classical MHC-I, ANGPTLs (e.g., ANGPTL2), CSPs, SEMA4A and CD1d;
d) is capable of specifically binding to LILRB2 with a binding affinity as measured by bio-layer interferometry of less than 20 nM (e.g., about 18 nM, 15 nM, 10 nM, 5 nM, 2 nM, 1.5 nM, about 1.4 nM, about 1.3 nM, about 1.2 nM, about 1.1 nM or about 1.0 nM); or
e) has an off-rate after binding to LILRB2 at a koff value of less than 5×10⁻⁴ (e.g., about 4×10⁻⁴, about 3×10⁻⁴)/s as measured by bio-layer interferometry.

6. The method of claim 5, wherein the anti-LILRB2 antibody comprises the same heavy chain complementary determining regions (HCDRs) and the same light chain CDRs (LCDRs) as the HCDRs and the LCDRs of the anti-LILRB2 antibody B2-19-16, wherein the anti-LILRB2 antibody B2-19-16 comprises a heavy chain variable region comprising the amino acid sequence of SEQ ID NO: 7, and a light chain variable region comprising the amino acid sequence of SEQ ID NO: 8.

7. The method of claim 6, wherein the anti-LILRB2 antibody comprises a heavy chain variable region comprising the amino acid sequence of SEQ ID NO:7, and a light chain variable region comprising the amino acid sequence of SEQ ID NO: 8.

8. The method of claim 6, wherein the anti-LILRB2 antibody comprises a heavy chain variable region comprising HCDR1, HCDR2 and HCDR3, and a light chain variable region comprising LCDR1, LCDR2 and LCDR3, wherein the HCDR1 comprises an amino acid sequence of SEQ ID NO: 1, the HCDR2 comprises an amino acid sequence of SEQ ID NO: 2, the HCDR3 comprises an amino acid sequence of SEQ ID NO: 3, the LCDR1 comprises an amino acid sequence of SEQ ID NO: 4, the LCDR2 comprises an amino acid sequence of SEQ ID NO: 5, and the LCDR3 comprises an amino acid sequence of SEQ ID NO: 6.

9. The method of any of claims 3-8, wherein the anti-LILRB2 antibody is administered at a dosage of about 600 mg to about 1800 mg Q3W, about 800 mg to about 1600 mg Q3W, about 1000 mg to about 1400 mg Q3W, or about 1200 mg Q3W.

10. The method of claim 3, wherein the anti-LILRB4 antibody:
a) comprises a constant region of human IgG1 with a functional Fc;
b) comprises a constant region of human IgG4;
b) is capable of binding to human LILRB4 at an IC50 value of less than 1 nM (e.g., less than 0.9 nM, less than 0.8 nM, less than 0.7 nM, less than 0.6 nM, less than 0.5 nM, less than 0.4 nM, less than 0.3 nM, less than 0.2 nM or less than 0.1 nM) as measured by flow cytometry;
c) is capable of blocking APOE binding to LILRB4 at an IC50 value of less than 0.5 nM (e.g., less than 0.4 nM, less than 0.3 nM, less than 0.2 nM or less than 0.1 nM) as measured by flow cytometry; or
d) is capable of blocking Fibronectin binding to LILRB4 at an IC50 value of less than 1 nM (e.g., less than 0.9 nM, less than 0.8 nM, less than 0.7 nM, less than 0.6 nM, less than 0.5 nM, less than 0.4 nM, less than 0.3 nM, less than 0.2 nM or less than 0.1 nM) as measured by flow cytometry.

11. The method of claim 10, wherein the anti-LILRB4 antibody comprises the same heavy chain complementary determining regions (HCDRs) and the same light chain CDRs (LCDRs) as the HCDRs and the LCDRs of the anti-LILRB4 antibody H7K3m5, wherein the anti-LILRB4 antibody H7K3m5 comprises a heavy chain variable region comprising the amino acid sequence of SEQ ID NO: 15, and a light chain variable region comprising the amino acid sequence of SEQ ID NO: 16.

12. The method of claim 11, wherein the anti-LILRB4 antibody comprises a heavy chain variable region comprising the amino acid sequence of SEQ ID NO: 15, and a light chain variable region comprising the amino acid sequence of SEQ ID NO: 16.

13. The method of claim 11, wherein the anti-LILRB4 antibody comprises a heavy chain variable region comprising HCDR1, HCDR2 and HCDR3, and a light chain variable region comprising LCDR1, LCDR2 and LCDR3, wherein the HCDR1 comprises an amino acid sequence of SEQ ID NO: 9, the HCDR2 comprises an amino acid sequence of SEQ ID NO: 10, the HCDR3 comprises an amino acid sequence of SEQ ID NO: 11, the LCDR1 comprises an amino acid sequence of SEQ ID NO: 12, the LCDR2 comprises an amino acid sequence of SEQ ID NO: 13, and the LCDR3 comprises an amino acid sequence of SEQ ID NO: 14.

14. The method of any one of claims 3 and 10-13, wherein the anti-LILRB4 antibody is administered Q3W at a dosage of about 250 mg to about 2400 mg, about 300 mg to about 2200 mg, about 350 mg to about 2000 mg, about 400 mg to about 1800 mg, about 450 mg to about 1600 mg, about 500 mg to about 1400 mg, about 550 mg to about 1200 mg, about 600 mg to about 1000 mg, about 650 mg to about 800 mg, or about 700 mg, with any dosing interval of once a week to once every 12-weeks (Q1W, Q2W, Q3W, Q4W, Q5W, Q6W, Q7W, Q8W, Q9W, Q10W, Q11W or Q12W), or once a month to once every 3 months (Q1M, Q2M or Q3M), or once quarterly.

15. The method of any one of claims 2-14, wherein the PD-1/PD-L1 axis inhibitor is a PD-1 inhibitor.

16. The method of claim 15, wherein the PD-1 inhibitor is selected from Table 1 or Table 2.

17. The method of claim 15, wherein the PD-1 inhibitor is pembrolizumab, cemiplimab, or tislelizumab.

18. The method of any one of claims 2-14, wherein the PD-1/PD-L1 axis inhibitor is a PD-L1 inhibitor.

19. The method of claim 18, wherein the PD-L1 inhibitor is selected from Table 3 or Table 4.

20. The method of claim 18, wherein the PD-L1 inhibitor is atezolizumab, durvalumab or avelumab.

21. The method of any of the preceding claims, wherein the subject is human.

22. The method of any of the preceding claims, wherein the administration is via oral, nasal, intravenous, subcutaneous, sublingual, intra-tumoral or intramuscular administration.

23. The method of any of the preceding claims, wherein the administration of the composition comprising an anti-LILRB2 antibody is prior to, simultaneously with, or after the administration of the composition comprising a PD-1/PD-L1 axis inhibitor.

24. The method of any of the preceding claims, wherein the disease or condition is cancer.

25. The method of claim 23, wherein the cancer is selected from the group consisting of: gastric cancer, lung cancer (e.g., non-small-cell lung carcinoma (NSCLC)), bronchial cancer, bone cancer, bile duct cancer, cholangiocarcinoma, pancreatic cancer, breast cancer (e.g., ER+HER2− breast cancer, triple-negative breast cancer (TNBC)), liver cancer (e.g., HCC), ovarian cancer, testicle cancer, kidney cancer (e.g., renal cell carcinoma (RCC), such as clear cell renal cell carcinoma (ccRCC)), bladder cancer, head and neck cancer (e.g., Head and neck squamous cell carcinoma (HNSCC)), nasopharyngeal cancer, spine cancer, brain cancer, cervix cancer, uterine cancer, endometrial cancer, colon cancer (e.g., MSI-H cancer), colon neuroendocrine, rectal cancer, anal cancer, esophageal cancer (e.g., esophageal squamous cell carcinoma (ESCC)), gastrointestinal cancer, Merkel cell carcinoma, skin cancer, prostate cancer, pituitary cancer, stomach cancer, vagina cancer, thyroid cancer, adrenal carcinoid tumor, glioblastoma, astrocytoma, melanoma, disseminated cancer unknown primary cancer, myelodysplastic syndrome, sarcoma, teratoma, adenocarcinoma and high microsatellite instability (MSI-H)/mismatch repair deficient (dMMR) tumors (e.g., MSI-H/dMMR CRC).

26. The method of claim 25, wherein the cancer is selected from the group consisting of: Merkel cell carcinoma, cholangiocarcinoma, colon neuroendocrine, rectal cancer, colon cancer, pancreatic cancer, HNSCC, breast cancer (e.g., ER+Her2− breast cancer), adrenal carcinoid tumor, bladder cancer, ovarian cancer, disseminated cancer unknown primary cancer, MSI-H/dMMR CRC, RCC, gastric cancer, NSCLC, TNBC and small intestine cancer.

27. A kit useful in treating a disease or condition in a subject in need thereof, comprising a first container that comprises an antagonist of LILRB and a second container that comprises a PD-1/PD-L1 axis inhibitor, and optionally instructions for use of the kit.

28. A kit, comprising an antagonist of LILRB and a package insert comprising instructions for using the antagonist of LILRB in combination with a PD-1/PD-L1 axis inhibitor to treat a disease or condition in a subject in need thereof.

29. Use of a pharmaceutical composition, comprising a therapeutically effective amount of a) an antagonist of LILRB, b) a PD-1/PD-L1 axis inhibitor, or c) both, and one or more pharmaceutically acceptable carriers, in the manufacture of a medicament for treating a disease or condition in a subject in need thereof.


SEQUENCE LISTING (22 sequences)

SEQ ID NO: 1 - protein, 8 aa, unidentified
GFTFSNAW

SEQ ID NO: 2 - protein, 10 aa, unidentified
IKSKTDTGTT

SEQ ID NO: 3 - protein, 16 aa, unidentified
TTDRYSSSWYSPAFDI

SEQ ID NO: 4 - protein, 8 aa, unidentified
SSNIGENV

SEQ ID NO: 5 - skipped

SEQ ID NO: 6 - protein, 11 aa, unidentified
ATWDESLSGPV

SEQ ID NO: 7 - protein, 125 aa, unidentified
EVQLVESGGGLVKPGGSLRLSCAASGFTFSNAWMSWVRQAPGKGLEWVGRIKSKTDTGTTDYAAPVKGRFTISRDDSKNTLYLQMNSLKTEDTAVYYCTTDRYSSSWYSPAFDIWGQGTTVSVSS

SEQ ID NO: 8 - protein, 110 aa, unidentified
QSVLTQPPSVSEAPRQRVTISCSGSSSNIGENVVNWYQQLPGKAPKLLIYYEDLLPSGVSDRFSGSKSGTSASLAISGLQSEDEADYYCATWDESLSGPVFGGGTKLTVL

SEQ ID NO: 9 - protein, 11 aa, unidentified
GFSLSSSYWIS

SEQ ID NO: 10 - protein, 21 aa, unidentified
WIGSIDSGSVGITYYATWVKG

SEQ ID NO: 11 - protein, 11 aa, unidentified
ARHGDNWALDL

SEQ ID NO: 12 - protein, 13 aa, unidentified
RASQSISSWLAWY

SEQ ID NO: 13 - protein, 11 aa, unidentified
LLIYKASTLAS

SEQ ID NO: 14 - protein, 12 aa, unidentified
QHGYIRGDLDNV

SEQ ID NO: 15 - protein, 120 aa, unidentified
EVQLVESGGGLVQPGGSLRLSCAASGFSLSSSYWISWVRQAPGKGLEWIGSIDSGSVGITYYATWVKGRFTISRDNSKNTLYLQMNSLRAEDTAVYYCARHGDNWALDLWGQGTLVTVSS

SEQ ID NO: 16 - protein, 110 aa, unidentified
DIQMTQSPSTLSASVGDRVTITCRASQSISSWLAWYQQKPGKAPKLLIYKASTLASGVPSRFSGSGSGTEFTLTISSLQPDDFATYYCQHGYIRGDLDNVFGGGTKVEIK

SEQ ID NO: 17 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 22 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC